(19) United States
(12) Patent Application Publication
(10) Pub. No.: US 2025/0306042 A1
(43) Pub. Date: Oct. 2, 2025

(54) METHOD FOR DIAGNOSING ENDOMETRIOSIS IN A SUBJECT

(71) Applicants: UNIVERZA V LJUBLJANI, Ljubgana (SI); Medizinische Universität Wien, Wien (AU); Helmholtz Zentrum München Deutsches Forschungszentrum für Gesundheit und Umwelt GmbH, Neuherberg (DE)

(72) Inventors: Tea Lanisnik Rizner, Ljubljana (SI); Andrej Vogler, Ljubljana (SI); Tamara Knific, Ljubljana (SI); René Wenzl, Ljubljana (SI); Jerzy Adamski, Neuherberg (DE); Alexander Cecil, Neuherberg (DE); Cornelia Prehn, Neuherberg (DE); Katja Vouk, Neuherberg (DE)

(21) Appl. No.: 18/844,571

(22) PCT Filed: Mar. 7, 2023
(86) PCT No.: PCT/EP2023/055709

§ 371 Date: Sep. 6, 2024

(30) Foreign Application Priority Data
Mar. 7, 2022 (LU) 501596

(51) Int. Cl. G01N 33/92; G01N 33/68

(52) U.S. Cl. CPC G01N 33/92; G01N 33/6812; G01N 2800/364

(57) ABSTRACT

The present invention generally relates to the use of metabolic biomarkers for the diagnosis of endometriosis, and more specifically to an ex vivo method for diagnosing endometriosis in a subject. The present invention further relates to a system and kit for diagnosing endometriosis.

### TECHNICAL FIELD OF THE INVENTION

The present invention generally relates to the use of a panel of metabolic biomarkers for the diagnosis of endometriosis, and more specifically to an ex vivo method for diagnosing endometriosis in a subject.

### BACKGROUND OF THE INVENTION

Endometriosis (ICD-10 N80) is a complex, benign neoplastic, gynecological disease with ectopic growth of endometrium-like tissue that affects around 170 million women worldwide; around 40,000 new cases are observed annually only in Germany. It manifests itself with dysmenorrhea, dyspareunia, increased risk of systemic or local inflammation, and chronic pelvic pain up to infertility (1, 2, 43). There are three main types of endometriosis: peritoneal endometriosis, ovarian endometriosis and deep infiltrating endometriosis depending on different location of ectopic endometrial tissue in the peritoneal cavity. The endometriosis can be as well manifested in a mixed form e.g. peritoneal and ovarian. Diagnosis is currently always invasive (with possible complications) using laparoscopy and subsequent histological analyzes (3, 4). Treatment for pain relief, prevention of recurrence, and maintenance of fertility includes pain killers and hormonal approaches (5, 6). Due to the high individual variability and unspecific symptoms, which can also be related to other diseases, it takes an average of seven years before endometriosis is finally diagnosed (6, 7). Apart from the diagnostic difficulties mentioned above, there are currently no reliable biomarkers that could predict the presence of endometriosis with high sensitivity and specificity (8, 9).

### Present Diagnostic Procedures for Endometriosis

The current gold standard in the present diagnostics is an invasive laparoscopy followed by histochemical analyses for pathology verification (10, 11). The laparoscopy may cause complications (e.g. infections or internal bleeding), is expensive, laborious (needs weeks to months for the communication of final outcome), requires adequate and certified training of participating physicians and pathologist. Sole laparoscopic examination without histological verification of pathology was not recommended in the clinical diagnostic routine (12). Analyses of accuracy of laparoscopy-based diagnosis demonstrated a huge need for new biomarkers (13, 14).

Noninvasive methods like ultrasound and Magnetic Resonance Imaging (MRI) have been checked for applicability to diagnostics as noninvasive approaches despite their huge hardware requirements. Ultrasound and 3D-ultrasound approaches were tested and found be applicable only to advanced stages of endometriosis. In the disease stages I-II and the III-IV the Area Under the Curve (AUC) was 0.68 and 0.84, respectively, but the methods were ranked as inadequate in routine diagnosis due to significant variability in the operator-dependent specificity and sensitivity (21, 22). The MRI analyses applied to detection of pelvic endometriosis suffered from the same issues in radiologists training. The MRI was found useful in diagnosing endometrial lesions with high specificity but poor sensitivity (23) and consequently not recommended as a replacement for laparoscopy (24).

### Health Care Costs

Some aspects, referred as indirect costs, cannot be directly calculated like that including loss of life quality due to pelvic pain, inflammation complications or infertility (15). The direct costs such as inpatient, outpatient, surgery, drug and other healthcare service vary among countries due to applied cost refund model. Indirect costs of endometriosis related to lost productivity at work ranged from $3,314 per patient per year in Austria (16) to $15,737 per patient per year in the USA (16) and $17,484 per patient per year in Australia (17). Productivity loss was depicted as around 6,298€ per woman per year affected in Europe (18). The diagnostic golden standard (laparoscopy) is around $3,313 (19). Ultrasound- and MRI-diagnostics is much more expensive than that by laparosopy. Long delays in diagnosis of endometriosis may cause up to 34,600 USD all-cause costs (20).

### Search for New Diagnostic Procedures

Plasma miRNA (hsa-miR-125b-5p, hsa-miR-28-5p and hsa-miR-29a-3p) was found to detect endometriosis in infertile woman with AUC of 0.60 and not further recommended (25). Several peptides and proteins or antigens present in serum were intensively tested for diagnostics performance. Serum miR-17, IL-4, and IL-6 reveal remarkable AUC of 0.84 in early stages of endometriosis (26) but they are quite unspecific and may reflect inflammatory processes of other origin. A similar issue was found for BDNF (brain-derived neurotrophic factor) which is highly elevated in endometrial tissue (27). The issue is that the BDNF could be as well elevated in structural brain pathology, depression, or persistent nociception (28) or hypoxia (29). The ovarian carcinoma biomarker CA-125 was repurposed for the endometriosis diagnostics but was found to be increased significantly only in stages III-IV with sensitivity of 46% at specificity of 89% and highly variable AUC in different cohorts (30). A combination of serum D-dimer, CA125 and data on neutrophil-to-lymphocyte ratio performed extremely well for the diagnostics of ovarian cancer (AUC 0.96) but not for the endometriosis (31). Genomic-approaches were so far unsuccessful in finding a single or a combination of genetic feature like methylation markers explaining endometriosis (32-34).

In past research for diagnostic biomarkers of endometriosis, WO2013/178794 studied a single indication of ovarian endometriosis only. In the particular cohort studied it was discovered that metabolite ratios perform far better than single reference values of concentrations (44). It was found that eight lipid metabolites were endometriosis-associated biomarkers due to elevated levels in patients compared with controls. A model containing hydroxysphingomyelin SMOH C16:1 and the ratio between phosphatidylcholine PCaa C36:2 to ether-phospholipid PCae C34:2, adjusted for the effect of age and the BMI, resulted in a sensitivity of 90.0%, a specificity of 84.3% and a ratio of the positive likelihood ratio to the negative likelihood ratio of 48.3. However, this discovery and the associated patent addressed only a single indication of ovarian endometriosis. The later is usually co-discovered in the invasive treatment of ovary and oviduct disorders. Furthermore, the proposed diagnostic model was based on ratio of two metabolites only.

### The Unmet Medical Need

In several documented applications the golden standard procedures do not have very high diagnostic performances as described by the AUC, sensitivity or specificity, further by positive predictive value or negative predictive value (35). Despite its wide use the AUC was judged as unreliable measure of screening performance because in practice the standard deviation of a screening or diagnostic test in affected and unaffected individuals can differ and instead detection rate (or sensitivity) and specificity should be used (36). For early cancer diagnostics the specificity, sensitivity or AUC the golden standard diagnostics markers might be really poorly performing but are used because of lack of alternatives in these frequent human disorders.

So far reference values established for different molecular biomarkers like DNA-variants, miRNA, protein or metabolite concentrations were unsuccessful in the clinical practice and never entered clinical routine. WO 2013/178794 addresses a diagnosis of ovarian endometriosis only (sole one form of endometriosis) and was not very attractive to the diagnostic market. The pressing unsolved issue is a procedure for unbiased detection of endometriosis types like peritoneal endometriosis and deep infiltrating endometriosis especially for patients where the endometriosis was not presumed at the first visit or based on unspecific symptoms. Thus, there remains a significant need to provide innovative methods and means for a cheap, fast, reliable and accurate diagnostic of endometriosis in a subject, notably a human female. Early and unbiased diagnostics of endometriosis would facilitate early hormonal or palliative therapies improving female health.

### SUMMARY OF THE INVENTION

The present invention is based on the identification and use of a panel of metabolic biomarkers for the diagnosis of endometriosis. However, instead of comparing to reference values in healthy individuals, the present invention uses selected multiple metabolite ratios. Different combinations of metabolite combinations like two predictors (two pairs of two metabolites) and three predictors (three pairs of two metabolites, example is provided in Table 1) were tested for diagnostic performance, and this was surprisingly successful in biostatistical evaluations. This approach has the huge advantage of its insensitivity to human metabolome variability caused by confounders like ethnicity, age, nutrition, lifestyle or medication. The metabolite-based diagnosis method of the present invention provides for a cheap, fast, reliable and accurate way for diagnosing endometriosis in a subject (the diagnostic flow scheme is described in FIGS. 1 and 2).

The present inventor's findings further reveal the potential for the combination of individual metabolite ratios to provide biomarkers for semi-invasive diagnostics. Moreover, the combination of at least two pairs of metabolites, and more specifically the combination of metabolite ratios thereof, allow distinction of endometriosis from control cases and can be used in the diagnostics of this disease, and are independent of age, BMI and menstrual cycle.

The present invention thus provides in a first aspect the use of a combination of at least two pairs, preferably at least three pairs, of metabolic biomarkers for the diagnosis of endometriosis and/or any sub-type thereof in a subject. In the present invention abbreviations of metabolite names are used which are identifiable by their abbreviations or synonymes as defined in the Table 2 and are known to experts in the field. As there are no diagnostic relevant metabolite concentration reference values established in large human clinical studies for endometriosis, the metabolite ratios and their absolute values in diseased women are compared to that of control samples. The diagnosis is based of calculation of values according to models. For each medical indication only one example is given. The example of use of metabolites in ratio composition may look like this (example taken from FIG. 4):
metabolite1/metabolite2+metabolite3/metabolite4+metabolite 5/metabolite6
or using metabolite abbreviations:
LysoPC a C17:0/SM(OH) C16:1+Arg/PC ae 36:0+PC ae C38:0/PC ae C40:0

An overview of indications covered and the corresponding exemplary metabolite ratio compositions used for a diagnostic analysis is shown in table 1. There are further possible distinct metabolite ratio models for each specific type of endometriosis (specific medical indications) but only those with best AUC will be described in detail later. The number of metabolite combinations used is limited by the AUC threshold, i.e. not all possible metabolite combinations would pass biostatistics evaluation for selectivity and sensitivity calculated for AUC. All metabolite combinations with AUC close to 0.5 or less have no diagnostic value and are not listed.

TABLE 1
Examples of metabolite ratio compositions for best diagnostic performance.

| | | | Value | Value | Log2 Fold |
|---|---|---|---|---|---|
| Endometriosis | Example of GLM model | | for | for | change |
| Indication | formula | AUC | control | case | observed |
| All types | LysoPC a C17:0_div_by_SM(OH) | 0.72 | 114.07 | 99.48 | 0.20 |
| | C16:1 + Arg_div_by_PC | | | | |
| | ae C36:0 + PC ae | | | | |
| | C38:0_div_by_PC ae C40:0 | | | | |
| Peritoneal | Thr_div_by_SM(OH) C22:2 + | 0.83 | 28.99 | 36.30 | −0.32 |
| | PC aa C40:5_div_by_SFA_PC + | | | | |
| | lysoPC a C16:0_div_by_SM(OH) | | | | |
| | C16:1 | | | | |
| Peritoneal | Orn_div_by_PC ae C38:0 + | 0.68 | 293.09 | 265.04 | 0.15 |
| mixed | C4_div_by_PC aa C38:4 + | | | | |
| | Tyr_div_by_PC aa C42:2 | | | | |
| Ovarian | PC aa C36:3_div_by_PC ae | 0.71 | 37.18 | 35.89 | 0.05 |
| | C40:5 + lysoPC a | | | | |
| | C14:0_div_by_PC aa C28:1 + | | | | |
| | Met_div_by_PC aa C36:3 | | | | |
| Ovarian mixed | C10_div_by_PC aa C36:6 + | 0.67 | 0.71 | 0.83 | −0.23 |
| | SM C20:2_div_by_PUFA_PC + | | | | |
| | PC ae C42:3_div_by_SM(OH) | | | | |
| | C16:1 | | | | |

GLM—generalized linear model, AUC—Area Under the Curve, metabolite abbreviations are explained in Table 2. Values for cases are calculated from concentrations of indicated metabolites according the model formula. A Log 2 fold change (numeric value, defined later as diagnostic score DxS) is calculated according to the used model. The calculated value is used to discriminate between diseased and not affected patient. Negative or positive values in fold change describe the direction of differences of case versus control.

Calculation of ROC and AUC with GLM Models and Cross-Validation of Models

As the classic statistical approach proved not to be robustly efficient the metabolite selection was performed by machine learning with randomForest (RF) on all metabolites and all possible metabolite ratios. All calculations are performed on the 10× cross validated data—this means data was randomly divided into 66% training data and 34% test data for each cross validation step. Therefore, every discovered model was validated in data not used for the creation of the model but in an independent data set. In order to narrow down the possible candidates for further modelling with GLM and to obtain reporter-operator curves (ROC) with area under the curve (AUC) calculations with restrictive parameters assuring robust diagnostic performance (described in detail later) were undertaken. From the remaining candidates only those in the top 10% of the performance were selected. In the following all possible combinations for 3-predictor model for the GLM approach were calculated. This results in 67599 possible combinations for these GLMs when leaving out metabolites/metabolite ratios which are derived total sums of measured metabolites. The later would be impractical to measure in a diagnostic assay and were excluded. The number of diagnostically relevant models is clearly limited by the AUC value which drops significantly if all combinations were included. Therefore only several models as listed later are relevant for diagnostics of each endometriosis indication. The GLMs were calculated on the response of samples being in the control group or case group. Although the ROCs with their respective AUCs shown in the following pages show an AUC up to average 0.82 in the test data set, it is still worth to note that it is very well possible to distinguish the responses in the models with a rather fair accuracy by selecting the parameters of the GLMs by RF from all the possible metabolites and ratios. This is not a feasible approach for PLS-DA analysis due to the high likelihood of over-fitting the model (FIG. 3). All results for cross-validation analyses of diagnostic models will be described for each medical indication in FIGS. 4-13.

### Concept of Diagnostic Flow

Samples are collected from patients using standard procedures in outpatient and inpatient stations (FIG. 1). Plasma is prepared and the metabolite analyses are undertaken with mass spectrometry apparatus. Data gained are undergoing processing with algorithm calculating values indicative of diagnostic status.

### Concept of Algorithm Implementation

The algorithm constitutes of calculation of GLM-values for distinct endometriosis forms. In particular, the calculation can be performed for:
Detection of any form of endometriosis
Detection of specific form like ovarian or peritoneal
Detection of mixed (multiple) forms like ovarian with coincidence of peritoneal and/or infiltrating

The algorithm can be implemented in parallel decision-making flow as depicted in FIG. 2.

More specifically, the present invention provides the use of a combination of at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH;
for the diagnosis of endometriosis and/or any sub-type thereof in a subject.

The present invention provides in a further aspect an ex vivo method of diagnosing endometriosis and/or any subtype thereof in a subject comprising quantifying in a sample obtained from said subject at least three pairs of metabolic biomarkers. More specifically, the present invention provides an ex vivo method of diagnosing endometriosis and/or any subtype thereof in a subject comprising a) quantifying in a sample obtained from said of at least two pairs, preferably at least three pairs, of metabolic biomarkers, determining the ratio for each of the at least two pairs and b) obtaining a diagnostic score using a generalized linear model (GLM). More specifically, the present invention provides an ex vivo method of diagnosing endometriosis and/or any subtype thereof in a subject, the method comprising
10) quantifying in a sample obtained from said subject at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH;
and b) obtaining a diagnostic score using a generalized linear model (GLM).

The present invention may be further characterized by the following items:
1. Use of a combination of at least two pairs, preferably at least three pairs, of metabolic biomarkers for the diagnosis of endometriosis and/or any sub-type thereof in a subject.
2. Use according to item 1, wherein a combination of at least three pairs of metabolic biomarkers for the diagnosis of endometriosis and/or any sub-type thereof in a subject, wherein the diagnosis involves use of the quantification of at least three pairs of metabolic biomarkers in a sample obtained from said subject.
3. Use according to item 1 or 2, wherein the at least two pairs, preferably at least three pairs, of metabolic biomarkers are selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH;
   for the diagnosis of endometriosis and/or any sub-type thereof.
4. The use according to any one of items 1 to 3, for diagnosing all endometriosis, wherein the at least two pairs, preferably at least three pairs, of metabolic biomarkers are selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Tyr and PC aa C42:4; C3-DC and C18; PC aa C42:1 and SM C22:3; and C6(C4:1-DC) and SM C16:1.
5. The use according to any one of items 1 to 3, for diagnosing peritoneal endometriosis, wherein the at least two pairs, preferably at least three pairs, of metabolic biomarkers are selected from the group of pairs consisting of Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; and PC ae C34:1 and PC ae C42:0.
6. The use according to any one of items 1 to 3, for diagnosing peritoneal mixed endometriosis, wherein the at least two pairs, preferably at least three pairs, of metabolic biomarkers are selected from the group of pairs consisting of Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; SM C18:0 and C5; Gly and SM C24:1; PC aa C32:0 and PC aa C40:1; PC aa C36:4 and PC aa C38:0; Gly and PC aa C42:5; and C0 and SM(OH) C22:2.
7. The use according to any one of items 1 to 3, for diagnosing ovarian endometriosis, wherein the at least two pairs, preferably at least three pairs, of metabolic biomarkers are selected from the group of pairs consisting of PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C0 and C5-M-DC; C3 and PC ae 34:0.
8. The use according to any one of items 1 to 3, for diagnosing ovarian mixed endometriosis, wherein the at least two pairs, preferably at least three pairs, of metabolic biomarkers are selected from the group of pairs consisting of C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; PC ae C44:3 and CPT I ratio; PC ae C34:0 and PC ae C40:3; C16:2-OH and SM C20:2; PC ae C44:6 and SM C22:3; and C10:1 and C14:2-OH.
9. The use according to any one of items 1 to 8, wherein the diagnosis involves use of a generalized linear model (GLM) based on the quantification of the at least two pairs, preferably at least three pairs, of metabolic biomarkers in a sample obtained from said subject.
10. The use according to item 9, wherein the generalized linear modelling (GLM) comprises i) determining the ratio of the concentrations for each of the at least two pairs, preferably at least three pairs, of metabolic biomarkers; and ii) calculating the sum of the obtained ratios (value for case).
11. The use according to item 10, wherein the generalized linear modelling (GLM) further comprises iii) obtaining a diagnostic score (DxS) calculated by forming the quotient between a predetermined reference value obtained from healthy subjects (value for control) and the sum of the obtained ratios (value for case)

$$DxS = log 2 ( \frac{predetermined reference value ( value for control )}{sum of the obtained ratios ( value for case )} )$$

Wherein said subject is diagnosed of having endometriosis or a sub-type thereof if the diagnostic score is different from zero (“0”), such as outside of the range 0±0.03.
12. An ex vivo method of diagnosing endometriosis and/or any subtype thereof in a subject comprising quantifying in a sample obtained from said subject at least three pairs of metabolic biomarkers.
13. The method according to item 12, which comprises quantifying in a sample obtained from said subject at least two pairs, preferably at least three pairs, of metabolic biomarkers.
14. The method according to item 12 or 13, wherein the at least two, preferably at least three pairs, of metabolic biomarkers are selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH.
15. The method according to item 13 or 14, wherein the generalized linear modelling comprises i) determining the ratio of the concentrations for each of the at least two pairs and ii) calculating the sum of the obtained ratios (value for case).
16. The method according to item 15, wherein the generalized linear modelling (GLM) further comprises iii) obtaining a diagnostic score (DxS) calculated by forming the quotient between a predetermined reference value obtained from healthy subjects (value for control) and the sum of the obtained ratios (value for case)

$$DxS = log 2 ( \frac{predetermined reference value ( value for control )}{sum of the obtained ratios ( value for case )} )$$

wherein said subject is diagnosed of having endometriosis or a sub-type thereof if the diagnostic score is different from zero (“0”) in the range 0±0.03. The DxS is enabling mathematic values obtained from calculations of metabolite ratios according to models (GLMs in the diagnostics). DxS values in the range of 0±0.03 are not facilitating diagnosis of specific indication of endometriosis type and other models have to be taken into the consideration as described in FIGS. 1 and 2.
17. The method according to any one of items 12 to 16, comprising determining whether the subject is suffering from any type of endometriosis.
18. The method according to item 17, comprising
   A1) quantifying in a sample obtained from said subject at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; and Tyr and PC aa C42:4; C3-DC and C18; PC aa C42:1 and SM C22:3; and C6(C4:1-DC) and SM C16:1; and
   B1) obtaining a diagnostic score using a generalized linear model (GLM).
19. The method according to item 17 or 18, comprising any one of the following procedures (1) to (15):
   (1) quantifying in a sample obtained from said subject the metabolites LysoPC a C17:0, SM(OH) C16:1, Arg, PC ae C36:0, PC ae C38:0 and PC ae C40:0; and performing GLM using the model formula LysoPC a C17:0_div_by_SM(OH) C16:1+Arg_div_by_PC ae C36:0+PC ae C38:0_div_by_PC ae C40:0;
   (2) quantifying in a sample obtained from said subject the metabolic biomarkers Arg, PC ae C36:0, LysoPC a C16:0, SM C18:1, PC ae C38:0 and PC ae C40:0; and performing GLM using the model formula Arg_div_by_PC ae C36:0+lysoPC a C16:0_div_by_SM C18:1+PC ae C38:0_div_by_PC ae C40:0;
   (3) quantifying in a sample obtained from said subject the metabolic biomarkers Thr, PC aa C34:3, LysoPC a C17:0, SM(OH) C16:1, Arg and PC ae C36:0; and performing GLM using the model formula Thr_div_by_PC aa C34:3+LysoPC a C17:0_div_by_SM(OH) C16:1+Arg_div_by_PC ae C36:0;
   (4) quantifying in a sample obtained from said subject the metabolic biomarkers Thr, PC aa C34:3, Arg, PC ae C36:0, LysoPC a C16:0 and SM C18:1; and performing GLM using the model formula Thr_div_by_PC aa C34:3+Arg_div_by_PC ae C36:0+lysoPC a C16:0_div_by_SM C18:1;
   (5) quantifying in a sample obtained from said subject the metabolic biomarkers Arg, PC aa C36:6, LysoPC a C17:0, SM(OH) C16:1, and PC ae C36:0; and performing GLM using the model formula Arg_div_by_PC aa C36:6+LysoPC a C17:0_div_by_SM(OH) C16:1+Arg_div_by_PC ae C36:0;
   (6) quantifying in a sample obtained from said subject the metabolic biomarkers LysoPC a C17:0, SM(OH) C16:1, Arg, PC ae C36:0, C18 and LysoPC a C14:0; and performing GLM using the model formula LysoPC a C17:0_div_by_SM(OH) C16:1+Arg_div_by_PC ae C36:0+C18_div_by_LysoPC a C14:0;
   (7) quantifying in a sample obtained from said subject the metabolic biomarkers LysoPC a C16:0, SM C18:1, PC ae C38:0, PC ae C40:0, Ser and PC ae C44:3; and performing GLM using the model formula LysoPC a C16:0_div_by_SM C18:1+PC ae C38:0_div_by_PC ae C40:0+Ser_div_by_PC ae C44:3;
   (8) quantifying in a sample obtained from said subject the metabolic biomarkers LysoPC a C17:0, SM(OH) C16:1, Arg, PC ae C36:0, Trp, PC ae C38:3; and performing GLM using the model formula LysoPC a C17:0_div_by_SM(OH) C16:1+Arg_div_by_PC ae C36:0+Trp_div_by_PC ae C38:3;
   (9) quantifying in a sample obtained from said subject the metabolic biomarkers Arg, PC ae C36:0, LysoPC a C16:0, SM C18:1, C8 and PC ae C30:0; and performing GLM using the model formula Arg_div_by_PC ae C36:0+LysoPC a C16:0_div_by_SM C18:1+C8_div_by_PC ae C30:0;
   (10) quantifying in a sample obtained from said subject the metabolic biomarkers Thr, PC ae C36:5, Arg, PC ae C36:0, LysoPC a C16:0 and SM C18:1; and performing GLM using the model formula Thr_div_by_PC ae C36:5+Arg_div_by_PC ae C36:0+LysoPC a C16:0_div_by_SM C18:1;
   (11) quantifying in a sample obtained from said subject the metabolite Arg, PC ae C36:0, C18, LysoPC a C14:0, LysoPC a C16:0 and SM C18:1; and performing GLM using the model formula Arg_div_by_PC ae C36:0+C18_div_by_LysoPC a C14:0+LysoPC a C16:0_div_by_SM C18:1;
   (12) quantifying in a sample obtained from said subject the metabolic biomarkers Arg, PC ae C36:0, C10, PC ae C38:6, LysoPC a C16:0 and SM C18:1; and performing GLM using the model formula Arg_div_by_PC ae C36:0+C10_div_by_PC ae C38:6+LysoPC a C16:0_div_by_SM C18:1;
   (13) quantifying in a sample obtained from said subject the metabolic biomarkers Arg, PC aa C36:6, PC ae C36:0, LysoPC a C16:0 and SM C18:1; and performing GLM using the model formula Arg_div_by_PC aa C36:6+Arg_div_by_PC ae C36:0+lysoPC a C16:0_div_by_SM C18:1;
   (14) quantifying in a sample obtained from said subject the metabolic biomarkers Tyr, PC aa C42:4, C3-DC, C18, PC aa C42:1 and SM C22:3; and performing GLM using the model formula Tyr_div_by_PC aa C42:4+C3-DC_div_by_C18+PC aa C42:1_div_by_SM C22:3;
   (15) quantifying in a sample obtained from said subject the metabolic biomarkers C3-DC, C18, PC aa C42:1, SM C22:3, C6(C4:1-DC) and SM C16:1; and performing GLM using the model formula C3-DC_div_by_C18+PC aa C42:1_div_by_SM C22:3+C6 (C4:1-DC)_div_by_SM C16:1.
20. The method according to any one of items 12 to 19, comprising determining whether the subject is suffering from peritoneal endometriosis.
21. The method according to item 20, comprising
   A2) quantifying in a sample obtained from said subject at least two pairs, preferably at least three pairs, of metabolic biomarkers selected from the group of pairs consisting of Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; and PC ae C34:1 and PC ae C42:0; and
   B2) obtaining a diagnostic score using a generalized linear model (GLM).
22. The method according to item 20 or 21, comprising any one of the following procedures (1) to (7):
   (1) quantifying in a sample obtained from said subject the metabolic biomarkers LysoPC a C16:0, SM(OH) C16:1, PC aa C32:0, SM C18:0, PC aa C32:0 and PC aa C38:3; and performing GLM using the model formula LysoPC a C16:0_div_by_SM(OH) C16:1+PC aa C32:0_div_by_SM C18:0+PC aa C32:0_div_by_PC aa C38:3;
   (2) quantifying in a sample obtained from said subject the metabolic biomarkers LysoPC a C16:0, SM(OH) C16:1, PC aa C32:0, SM C18:0, Arg and PC ae C34:0; and performing GLM using the model formula LysoPC a C16:0_div_by_SM(OH) C16:1+PC aa C32:0_div_by_SM C18:0+Arg_div_by_PC ae C34:0;
   (3) quantifying in a sample obtained from said subject the metabolic biomarkers C5-M-DC, PC aa C42:5, Arg, PC ae C34:0, LysoPC a C18:2 and PC ae C40:6; and performing GLM using the model formula C5-M-DC_div_by_PC aa C42:5+Arg_div_by_PC ae C34:0+LysoPC a C18:2_div_by_PC ae C40:6;
   (4) quantifying in a sample obtained from said subject the metabolic biomarkers LysoPC a C18:2, PC ae C40:4, PC ae C40:6, CPT I ratio, LysoPC a C17:0 and SM C18:0; and performing GLM using the model formula LysoPC a C18:2_div_by_PC ae C40:4+PC ae C40:6_div_by_CPT I ratio+lysoPC a C17:0_div_by_SM C18:0;
   (5) quantifying in a sample obtained from said subject the metabolic biomarkers C4, PC ae C30:2, Arg, PC ae C34:0, LysoPC a C18:2 and PC ae C40:6; and performing GLM using the model formula C4_div_by_PC ae C30:2+Arg_div_by_PC ae C34:0+LysoPC a C18:2_div_by_PC ae C40:6;
   (6) quantifying in a sample obtained from said subject the metabolic biomarkers PC ae C40:6, CPT I ratio, C4, PC ae C30:2, LysoPC a C18:2 and PC ae C40:6; and performing GLM using the model formula PC ae C40:6_div_by_CPT I ratio+C4_div_by_PC ae C30:2+LysoPC a C18:2_div_by_PC ae C40:6;
   (7) quantifying in a sample obtained from said subject the metabolic biomarkers LysoPC a C18:2, PC ae C40:4, Arg, PC ae C34:0, PC ae C34:1 and PC ae C42:0; and performing GLM using the model formula LysoPC a C18:2_div_by_PC ae C40:4+Arg_div_by_PC ae C34:0+PC ae C34:1_div_by_PC ae C42:0.
23. The method according to any one of items 12 to 22, comprising determining whether the subject is suffering from peritoneal mixed endometriosis.
24. The method according to item 23, comprising
   A3) quantifying in a sample obtained from said subject at least two pairs, preferably at least three pairs, of metabolic biomarkers selected from the group of pairs consisting of Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; Gly and SM C24:1; PC aa C32:0 and PC aa C40:1; PC aa C36:4 and PC aa C38:0; Gly and PC aa C42:5; and C0 and SM(OH) C22:2; and
   B3) performing generalized linear modelling (GLM).
25. The method according to item 23 or 24, comprising any one of the following procedures (1) to (16):
   (1) quantifying in a sample obtained from said subject the metabolic biomarkers Orn, PC ae C38:0, C4, PC aa C38:4, Tyr and PC aa C42:2; and performing GLM using the model formula Orn_div_by_PC ae C38:0+C4_div_by_PC aa C38:4+Tyr_div_by_PC aa C42:2;
   (2) quantifying in a sample obtained from said subject the metabolic biomarkers Arg, PC aa C36:6, C5 and LysoPC a C17:0; and performing GLM using the model formula Arg_div_by_PC aa C36:6+C5_div_by_lysoPC a C17:0+C5_div_by_Arg;
   (3) quantifying in a sample obtained from said subject the metabolic biomarkers Orn, PC ae C38:0, C5, LysoPC a C17:0 and Arg; and performing GLM using the model formula Orn_div_by_PC ae C38:0+C5_div_by_lysoPC a C17:0+C5_div_by_Arg;
   (4) quantifying in a sample obtained from said subject the metabolic biomarkers C0, Gly, Orn, PC ae C38:0, Tyr and PC aa C42:2; and performing GLM using the model formula C0_div_by_Gly+Orn_div_by_PC ae C38:0+Tyr_div_by_PC aa C42:2;
   (5) quantifying in a sample obtained from said subject the metabolic biomarkers SM C18:0, C5, LysoPC a C17:0 and Arg; and performing GLM using the model formula SM C18:0+C5_div_by_lysoPC a C17:0+C5_div_by_Arg;
   (6) quantifying in a sample obtained from said subject the metabolic biomarkers C5, LysoPC a C17:0, Arg, Ser and SM(OH) C16:1; and performing GLM using the model formula C5_div_by_lysoPC a C17:0+C5_div_by_Arg+Ser_div_by_SM(OH) C16:1;
   (7) quantifying in a sample obtained from said subject the metabolic biomarkers SM C18:0, C0, Gly, Tyr and PC aa C42:2; and performing GLM using the model formula SM C18:0+C0_div_by_Gly+Tyr_div_by_PC aa C42:2;
   (8) quantifying in a sample obtained from said subject the metabolic biomarkers Orn, PC ae C38:0, C3, PC ae C40:5, Tyr and PC aa C42:2; and performing GLM using the model formula Orn_div_by_PC ae C38:0+C3_div_by_PC ae C40:5+Tyr_div_by_PC aa C42:2;
   (9) quantifying in a sample obtained from said subject the metabolic biomarkers Pro, PC ae C34:0, Orn, PC ae C38:0, Tyr and PC aa C42:2; and performing GLM using the model formula Pro_div_by_PC ae C34:0+Orn_div_by_PC ae C38:0+Tyr_div_by_PC aa C42:2;
   (10) quantifying in a sample obtained from said subject the metabolic biomarkers C4, Ser, Orn, PC ae C38:0, Tyr and PC aa C42:2; and performing GLM using the model formula C4_div_by_Ser+Orn_div_by_PC ae C38:0+Tyr_div_by_PC aa C42:2;
   (11) quantifying in a sample obtained from said subject the metabolic biomarkers SM C18:0, Orn, PC ae C38:0, Tyr and PC aa C42:2; and performing GLM using the model formula SM C18:0+Orn_div_by_PC ae C38:0+Tyr_div_by_PC aa C42:2;
   (12) quantifying in a sample obtained from said subject the metabolic biomarkers Orn, PC ae C38:0, C4, PC ae C40:3, Tyr and PC aa C42:2; and performing GLM using the model formula Orn_div_by_PC ae C38:0+C4_div_by_PC ae C40:3+Tyr_div_by_PC aa C42:2;
   (13) quantifying in a sample obtained from said subject the metabolic biomarkers Orn, PC ae C38:0, PC ae C42:3, SM(OH) C16:1, Tyr and PC aa C42:2; and performing GLM using the model formula Orn_div_by_PC ae C38:0+PC ae C42:3_div_by_SM(OH) C16:1+Tyr_div_by_PC aa C42:2;
   (14) quantifying in a sample obtained from said subject the metabolic biomarkers Orn, PC ae C38:0, Tyr, PC ae C38:0 and PC aa C42:2; and performing GLM using the model formula Orn_div_by_PC ae C38:0+Tyr_div_by_PC ae C38:0+Tyr_div_by_PC aa C42:2;
   (15) quantifying in a sample obtained from said subject the metabolic biomarkers Gly, SM C24:1, PC aa C32:0, PC aa C40:1, PC aa C36:4 and PC aa C38:0; and performing GLM using the model formula Gly_div_by_SM C24:1+PC aa C32:0_div_by_PC aa C40:1+PC aa C36:4_div_by_PC aa C38:0;
   (16) quantifying in a sample obtained from said subject the metabolic biomarkers Gly, PC aa C42:5, PC aa C36:4, PC aa C38:0SM, C0 and SM(OH) C22:2; and performing GLM using the model formula Gly_div_by_PC aa C42:5+PC aa C36:4_div_by_PC aa C38:0+C0_div_by_SM(OH) C22:2.
26. The method according to any one of items 12 to 25, comprising determining whether the subject is suffering from ovarian endometriosis.
27. The method according to item 26, comprising
   A4) quantifying in a sample obtained from said subject at least two pairs, preferably at least three pairs, of metabolic biomarkers selected from the group of pairs consisting of PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; and LysoPC a C20:4 and PC aa C32:3; and
   B4) performing generalized linear modelling (GLM).
28. The method according to item 26 or 27, comprising any one of the following procedures (1) to (14):
   (1) quantifying in a sample obtained from said subject the metabolic biomarkers PC aa C36:3, PC ae C40:5, LysoPC a C14:0, PC aa C28:1 and Met; and performing GLM using the model formula PC aa C36:3_div_by_PC ae C40:5+LysoPC a C14:0_div_by_PC aa C28:1+Met_div_by_PC aa C36:3;
   (2) quantifying in a sample obtained from said subject the metabolic biomarkers PC aa C38:0, PC ae C36:1, Thr, SM (OH) C22:1, LysoPC a C14:0 and PC aa C28:1; and performing GLM using the model formula PC aa C38:0_div_by_PC ae C36:1+Thr_div_by_SM (OH) C22:1+lysoPC a C14:0_div_by_PC aa C28:1;
   (3) quantifying in a sample obtained from said subject the metabolic biomarkers Thr, SM (OH) C22:1, PC aa C28:1, PC ae C34:3, C18:2 and PC ae C34:3; and performing GLM using the model formula Thr_div_by_SM (OH) C22:1+PC aa C28:1_div_by_PC ae C34:3+C18:2_div_by_PC ae C34:3;
   (4) quantifying in a sample obtained from said subject the metabolic biomarkers PC aa C36:3, PC ae C40:5, C3, PC ae C34:1, Met and PC aa C36:3; and performing GLM using the model formula PC aa C36:3_div_by_PC ae C40:5+C3_div_by_PC ae C34:1+Met_div_by_PC aa C36:3;
   (5) quantifying in a sample obtained from said subject the metabolic biomarkers PC aa C36:3, PC ae C40:5, PC aa C28:1, PC ae C34:3, Gly and PC ae C36:1; and performing GLM using the model formula PC aa C36:3_div_by_PC ae C40:5+PC aa C28:1_div_by_PC ae C34:3+Gly_div_by_PC ae C36:1;
   (6) quantifying in a sample obtained from said subject the metabolic biomarkers PC aa C38:0, PC ae C36:1, C18:2, PC ae C34:3, Met and PC aa C36:3; and performing GLM using the model formula PC aa C38:0_div_by_PC ae C36:1+C18:2_div_by_PC ae C34:3+Met_div_by_PC aa C36:3;
   (7) quantifying in a sample obtained from said subject the metabolic biomarkers PC aa C38:0, PC ae C36:1, C10:1, PC aa C36:1, PC ae C38:3 and SM C18:1; and performing GLM using the model formula PC aa C38:0_div_by_PC ae C36:1+C10:1_div_by_PC aa C36:1+PC ae C38:3_div_by_SM C18:1;
   (8) quantifying in a sample obtained from said subject the metabolic biomarkers PC aa C38:0, PC ae C36:1, Gly, C3 and PC ae C34:1; and performing GLM using the model formula PC aa C38:0_div_by_PC ae C36:1+Gly_div_by_PC ae C36:1+C3_div_by_PC ae C34:1;
   (9) quantifying in a sample obtained from said subject the metabolic biomarkers PC aa C38:0, PC ae C36:1, C12-DC, C14:2, PC ae C38:3 and SM C18:1; and performing GLM using the model formula PC aa C38:0_div_by_PC ae C36:1+C12-DC_div_by_C14:2+PC ae C38:3_div_by_SM C18:1;
   (10) quantifying in a sample obtained from said subject the metabolic biomarkers PC aa C36:3, PC ae C40:5, PC aa C38:3, PC ae C44:5 and Met; and performing GLM using the model formula PC aa C36:3_div_by_PC ae C40:5+PC aa C38:3_div_by_PC ae C44:5+Met_div_by_PC aa C36:3;
   (11) quantifying in a sample obtained from said subject the metabolic biomarkers PC aa C38:0, PC ae C36:1, PC ae C38:3, SM C18:1, Met and PC aa C36:3; and performing GLM using the model formula PC aa C38:0_div_by_PC ae C36:1+PC ae C38:3_div_by_SM C18:1+Met_div_by_PC aa C36:3;
   (12) quantifying in a sample obtained from said subject the metabolic biomarkers PC aa C28:1, PC ae C34:3, C18:2, PC ae C34:3, C4 and C5:1; and performing GLM using the model formula PC aa C28:1_div_by_PC ae C34:3+C18:2_div_by_PC ae C34:3+C4_div_by_C5:1;
   (13) quantifying in a sample obtained from said subject the metabolic biomarkers PC aa C36:3, PC ae C40:5, LysoPC a C20:4, PC ae C32:1 and Met; and performing GLM using the model formula PC aa C36:3_div_by_PC ae C40:5+lysoPC a C20:4_div_by_PC ae C32:1+Met_div_by_PC aa C36:3;
   (14) quantifying in a sample obtained from said subject the metabolic biomarkers PC aa C36:3, PC ae C40:5, LysoPC a C20:4, PC aa C32:3 and Met; and performing GLM using the model formula PC aa C36:3_div_by_PC ae C40:5+lysoPC a C20:4_div_by_PC aa C32:3+Met_div_by_PC aa C36:3.
29. The method according to any one of items 12 to 28, comprising determining whether the subject is suffering from ovarian mixed endometriosis.
30. The method according to item 29, comprising
   A5) quantifying in a sample obtained from said subject at least two pairs, preferably at least three pairs, of metabolic biomarkers selected from the group of pairs consisting of C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; PC ae C44:3 and CPT I ratio; PC ae C34:0 and PC ae C40:3; C16:2-OH and SM C20:2; PC ae C44:6 and SM C22:3; and C10:1 and C14:2-OH; and
   B5) performing generalized linear modelling (GLM).
31. The method according to item 23 or 24, comprising any one of the following procedures (1) to (13):
   (1) quantifying in a sample obtained from said subject the metabolic biomarkers C10, PC aa C36:6, Pro, PC ae C34:0, PC ae C42:3 and SM(OH) C16:1; and performing GLM using the model formula C10_div_by_PC aa C36:6+Pro_div_by_PC ae C34:0+PC ae C42:3_div_by_SM(OH) C16:1;
   (2) quantifying in a sample obtained from said subject the metabolic biomarkers C10, PC aa C36:6, PC ae C42:3, SM(OH) C16:1, C6:1 and LysoPC a C20:4; and performing GLM using the model formula C10_div_by_PC aa C36:6+PC ae C42:3_div_by_SM(OH) C16:1+C6:1_div_by_lysoPC a C20:4;
   (3) quantifying in a sample obtained from said subject the metabolic biomarkers C10, PC aa C36:6, PC ae C42:3, SM(OH) C16:1, LysoPC a C20:4 and PC ae C40:2; and performing GLM using the model formula C10_div_by_PC aa C36:6+PC ae C42:3_div_by_SM(OH) C16:1+lysoPC a C20:4_div_by_PC ae C40:2;
   (4) quantifying in a sample obtained from said subject the metabolic biomarkers Ser, PC aa C38:3, C10, PC aa C36:6, PC ae C42:3 and SM(OH) C16:1; and performing GLM using the model formula Ser_div_by_PC aa C38:3+C10_div_by_PC aa C36:6+PC ae C42:3_div_by_SM(OH) C16:1;
   (5) quantifying in a sample obtained from said subject the metabolic biomarkers C10, LysoPC a C18:1, PC aa C36:6, PC ae C42:3 and SM(OH) C16:1; and performing GLM using the model formula C10_div_by_lysoPC a C18:1+C10_div_by_PC aa C36:6+PC ae C42:3_div_by_SM(OH) C16:1;
   (6) quantifying in a sample obtained from said subject the metabolic biomarkers C10, PC aa C36:6, LysoPC a C24:0, PC ae C42:3, PC ae C42:3 and SM(OH) C16:1; and performing GLM using the model formula C10_div_by_PC aa C36:6+LysoPC a C24:0_div_by_PC ae C42:3+PC ae C42:3_div_by_SM(OH) C16:1;
   (7) quantifying in a sample obtained from said subject the metabolic biomarkers C10, PC aa C36:6, LysoPC a C18:1, PC aa C36:1, PC ae C42:3 and SM(OH) C16:1; and performing GLM using the model formula C10_div_by_PC aa C36:6+LysoPC a C18:1_div_by_PC aa C36:1+PC ae C42:3_div_by_SM(OH) C16:1;
   (8) quantifying in a sample obtained from said subject the metabolic biomarkers C10, PC aa C36:6, +Gly, PC ae C34:1, PC ae C42:3 and SM(OH) C16:1; and performing GLM using the model formula C10_div_by_PC aa C36:6+Gly_div_by_PC ae C34:1+PC ae C42:3_div_by_SM(OH) C16:1;
   (9) quantifying in a sample obtained from said subject the metabolic biomarkers Gln, PC ae C30:2, C10, PC aa C36:6, PC ae C42:3 and SM(OH) C16:1; and performing GLM using the model formula Gln_div_by_PC ae C30:2+C10_div_by_PC aa C36:6+PC ae C42:3_div_by_SM(OH) C16:1;
   (10) quantifying in a sample obtained from said subject the metabolic biomarkers Pro, PC ae C34:0, LysoPC a C24:0, PC ae C42:3, PC ae C42:3 and SM(OH) C16:1; and performing GLM using the model formula Pro_div_by_PC ae C34:0+lysoPC a C24:0_div_by_PC ae C42:3+PC ae C42:3_div_by_SM(OH) C16:1;
   (11) quantifying in a sample obtained from said subject the metabolic biomarkers C10:1, LysoPC a C24:0, LysoPC a C24:0, PC ae C42:3 and SM(OH) C16:1; and performing GLM using the model formula C10:1_div_by_lysoPC a C24:0+lysoPC a C24:0_div_by_PC ae C42:3+PC ae C42:3_div_by_SM(OH) C16:1;
   (12) quantifying in a sample obtained from said subject the metabolic biomarkers PC ae C44:3, CPT I ratio, PC ae C34:0, PC ae C40:3, C16:2-OH and SM C20:2; and performing GLM using the model formula PC ae C44:3_div_by_CPT.I.ratio+PC ae C34:0_div_by_PC ae C40:3+C16:2-OH_div_by_SM C20:2;
   (13) quantifying in a sample obtained from said subject the metabolic biomarkers PC ae C44:6, SM C22:3, PC ae C34:0, PC ae C40:3, C10:1 and C14:2-OH; and performing GLM using the model formula PC ae C44:6_div_by_SM C22:3+PC ae C34:0_div_by_PC ae C40:3+C10:1_div_by_C14:2-OH.
32. The method according to any one of items 12 to 31, wherein the sample is selected from blood, serum, plasma, saliva, urine, cerebrospinal fluid, condensates from respiratory air, tears, mucosal tissue, mucus, vaginal tissue, endometrium, eutopic endometrium, skin, hair and hair follicle.
33. The method according to any one of items 12 to 31, wherein the sample is selected from blood, serum and plasma.
34. The method according to any one of items 12 to 31, wherein the sample is plasma.
35. The use according to any one of items 1 to 11 or the method according to any one of items 12 to 34, wherein the subject is a human subject.
36. The use or method according to item 35, wherein the human subject is a female.
37. The use or method according to item 35 or 36, wherein the human subject is of Caucasian race.
38. The use or method according to any one of items 35 to 37, wherein the subject is suspected to suffer from endometriosis or to have a predisposition therefore.

### BRIEF DESCRIPTION OF THE FIGURES

FIG. 1: Process flow of diagnostic assay. Samples are collected from patients using standard procedures in outpatient and inpatient stations. Plasma is prepared and the metabolite analyses are undertaken with mass spectrometry apparatus. Data gained are undergoing processing with algorithm calculating values indicative of diagnostic status. DxS—calculated diagnostic score: log 2 (ratio of GLM of control and GLM of patient).

FIG. 2: Concept of algorithm implementation. The algorithm constitutes of calculation of GLM-values based on metabolite ratios measured in patient plasma. For distinct endometriosis forms different GLMs are indicative for the diagnosis. Should the DxS (ratio of GLM of control and GLM of patient) be zero these GLM can not be used for diagnosis and another GLM values are considered. All GLM models can be tested for the given sample in parallel. In particular, the calculation can be performed for: 1. Detection of any form of endometriosis, 2. Detection of specific form like ovarian or peritoneal, 3. Detection of mixed (multiple) forms like ovarian with coincidence of peritoneal and/or infiltrating. DxS—calculated diagnostic score.

FIG. 3: PLS-DA analysis for case vs control using absolute concentrations of metabolites. The calculated parameters indicate lack of separation according of this calculation: R_{γ}²=0.355, Rₓ²=0.445, Qₓ²=−0.592, RMSE=0.39, P_{R}²=−01515, P_{Q}²=−0.6585.

FIG. 4: Calculation of AUC for GLM model #1 for all cases of endometriosis for LysoPC a C17:0_div_by_SM(OH) C16:1+Arg_div_by_PC ae C36:0+PC ae C38:0_div_by_PC ae C40:0

FIG. 5: Calculation of AUC for GLM model #1 for all cases of endometriosis as composite plot with all test data sets (cross-validated) displayed for LysoPC a C17:0_div_by_SM(OH) C16:1+Arg_div_by_PC ae C36:0+PC ae C38:0_div_by_PC ae C40:0

FIG. 6: Calculation of AUC for GLM model #1 for peritoneal endometriosis—LysoPC a C16:0_div_by_SM(OH) C16:1+PC aa C32:0_div_by_SM C18:0+PC aa C32:0_div_by_PC aa C38:3

FIG. 7: Calculation of AUC for GLM model #1 peritoneal endometriosis as composite plot with all test data sets (cross-validated) displayed for LysoPC a C16:0_div_by_SM(OH) C16:1+PC aa C32:0_div_by_SM C18:0+PC aa C32:0_div_by_PC aa C38:3

FIG. 8: Calculation of AUC for GLM model #1 for peritoneal mixed endometriosis for Orn_div_by_PC ae C38:0+C4_div_by_PC aa C38:4+Tyr_div_by_PC aa C42:2

FIG. 9: Calculation of AUC for GLM model #1 for peritoneal mixed endometriosis as composite plot with all test data sets (cross-validated) displayed for Orn_div_by_PC ae C38:0+C4_div_by_PC aa C38:4+Tyr_div_by_PC aa C42:2

FIG. 10: Calculation of AUC for GLM model #1 for ovarian endometriosis for PC aa C36:3_div_by_PC ae C40:5+lysoPC a C14:0_div_by_PC aa C28:1+Met_div_by_PC aa C36:3

FIG. 11: Calculation of AUC for GLM model #1 for ovarian endometriosis as composite plot with all test data sets (cross-validated) displayed for PC aa C36:3_div_by_PC ae C40:5+lysoPC a C14:0_div_by_PC aa C28:1+Met_div_by_PC aa C36:3

FIG. 12: Calculation of AUC for GLM model #1 for ovarian mixed endometriosis for C10_div_by_PC aa C36:6+Pro_div_by_PC ae C34:0+PC ae C42:3_div_by_SM(OH) C16:1

FIG. 13: Calculation of AUC for GLM model #1 for ovarian mixed endometriosis as composite plot with all test data sets (cross-validated) displayed for: C10_div_by_PC aa C36:6+Pro_div_by_PC ae C34:0+PC ae C42:3_div_by_SM(OH) C16:1

The present invention is now described in more detail below.

### DETAILED DESCRIPTION OF THE INVENTION

As noted above, the present invention is based on the identification and use of a panel of metabolic biomarkers for the diagnosis of endometriosis. However, instead of comparing to reference values in healthy individuals, the present invention uses selected metabolite ratios. Different combinations of metabolite combinations like two predictors (two pairs of two metabolites) and three predictors (three pairs of two metabolites) were tested for diagnostic performance, and was successful in biostatistical evaluations. This approach has the huge advantage of its insensitivity to human metabolome variability caused by confounders like ethnicity, age, nutrition, lifestyle or medication. The metabolite-based diagnosis method of the present invention provides for a cheap, fast, reliable and accurate way for diagnosing endometriosis in a subject.

Specifically, the present inventors have identified the following pairs of metabolic biomarkers most suitable for the diagnosis of endometriosis and/or any sub-type thereof in a subject: LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; and PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH.

Besides have generally identified pairs of metabolic biomarkers most suitable for the diagnosis of endometriosis, the present inventors have identified various subgroups of these pairs of metabolic biomarkers which allow for the diagnosis of any form of endometriosis (all endometriosis), the diagnosis of a specific form like ovarian or peritoneal, and/or the diagnosis of mixed (multiple) forms like ovarian with coincidence of peritoneal and/or infiltrating.

Specifically, the following pairs of metabolic biomarkers have been shown to provide a diagnostic score for diagnosing all endometriosis: LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; and Tyr and PC aa C42:4; C3-DC and C18; PC aa C42:1 and SM C22:3; and C6(C4:1-DC) and SM C16:1.

The following pairs of metabolic biomarkers have been shown to provide a diagnostic score for diagnosing peritoneal endometriosis: Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; and PC ae C34:1 and PC ae C42:0.

The following pairs of metabolic biomarkers have been shown to provide a diagnostic score for diagnosing peritoneal mixed endometriosis: Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; SM C18:0 and C5; Gly and SM C24:1; PC aa C32:0 and PC aa C40:1; PC aa C36:4 and PC aa C38:0; Gly and PC aa C42:5; and C0 and SM(OH) C22:2.

The following pairs of metabolic biomarkers have been shown to provide a diagnostic score for diagnosing ovarian endometriosis: PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C0 and C5-M-DC; C3 and PC ae 34:0.

The following pairs of metabolic biomarkers have been shown to provide a diagnostic score for diagnosing ovarian mixed endometriosis: C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; PC ae C44:3 and CPT I ratio; PC ae C34:0 and PC ae C40:3; C16:2-OH and SM C20:2; PC ae C44:6 and SM C22:3; and C10:1 and C14:2-OH.

TABLE 2
Metabolites used in accordance with the invention for diagnosing endometriosis.

| | | | CAS | Chemical |
|---|---|---|---|---|
| Metabolite | Trivial name | HMDB ID | number | Formula |
| PC aa C28:1 | Phosphatidylcholine aa C28:1 | HMDB07867 | na | C36H70NO8P |
| PC ae C30:0 | Phosphatidylcholine ae C30:0 | HMDB13341 | na | C38H78NO7P |
| PC aa C32:0 | Phosphatidylcholine aa C32:0 | HMDB00564 | 63-89-8 | C40H80NO8P |
| PC aa C32:3 | Phosphatidylcholine aa C32:3 | HMDB07876 | na | C40H74NO8P |
| PC aa C34:3 | Phosphatidylcholine aa C34:3 | HMDB08006 | 182820- | C42H78NO8P |
| | | | 31-1 | |
| PC aa C36:1 | Phosphatidylcholine aa C36:1 | HMDB08037 | na | C44H86NO8P |
| PC aa C36:3 | Phosphatidylcholine aa C36:3 | HMDB07980 | na | C44H82NO8P |
| PC aa C36:4 | Phosphatidylcholine aa C36:4 | HMDB07982 | na | C44H80NO8P |
| PC aa C36:6 | Phosphatidylcholine aa C36:6 | HMDB07892 | na | C44H76NO8P |
| PC aa C38:0 | Phosphatidylcholine aa C38:0 | HMDB07893 | na | C46H92NO8P |
| PC aa C38:3 | Phosphatidylcholine aa C38:3 | HMDB08046 | na | C46H86NO8P |
| PC aa C38:4 | Phosphatidylcholine aa C38:4 | HMDB07988 | na | C46H84NO8P |
| PC aa C40:1 | Phosphatidylcholine aa C40:1 | HMDB13433 | na | C48H96NO7P |
| PC aa C40:4 | Phosphatidylcholine aa C40:4 | HMDB08054 | na | C48H88NO8P |
| PC aa C40:5 | Phosphatidylcholine aa C40:5 | HMDB08055 | na | C48H86NO8P |
| PC aa C42:1 | Phosphatidylcholine aa C42:1 | HMDB08059 | na | C50H98NO8P |
| PC aa C42:2 | Phosphatidylcholine aa C42:2 | HMDB08570 | na | C50H96NO8P |
| PC aa C42:4 | Phosphatidylcholine aa C42:4 | HMDB08572 | na | C50H92NO8P |
| PC aa C42:5 | Phosphatidylcholine aa C42:5 | HMDB08287 | na | C50H90NO8P |
| PC ae C30:2 | Phosphatidylcholine ae C30:2 | HMDB0013410 | na | C38H74NO7P |
| PC ae C32:1 | Phosphatidylcholine ae C32:1 | HMDB0007898 | na | C40H78NO7P |
| PC ae C34:0 | Phosphatidylcholine ae C34:0 | HMDB13405 | na | C42H86NO7P |
| PC ae C34:1 | Phosphatidylcholine ae C34:1 | HMDB0013412 | na | C42H84NO7P |
| PC ae C34:3 | Phosphatidylcholine ae C34:3 | HMDB0013413 | na | C42H80NO7P |
| PC ae C36:0 | Phosphatidylcholine ae C36:0 | HMDB13406 | na | C44H90NO7P |
| PC ae C36:1 | Phosphatidylcholine ae C36:1 | HMDB13427 | na | C44H88NO7P |
| PC ae C36:5 | Phosphatidylcholine ae C36:5 | HMDB11222 | na | C44H78NO7P |
| PC ae C38:0 | Phosphatidylcholine ae C38:0 | HMDB13408 | na | C46H94NO7P |
| PC ae C38:3 | Phosphatidylcholine ae C38:3 | HMDB13439 | na | C46H88NO7P |
| PC ae C38:6 | Phosphatidylcholine ae C38:6 | HMDB13409 | na | C46H82NO7P |
| PC ae C40:0 | Phosphatidylcholine ae C40:0 | HMDB13421 | na | C48H98NO7P |
| PC aa C40:1 | Phosphatidylcholine aa C40:1 | HMDB13433 | na | C48H96NO7P |
| PC ae C40:2 | Phosphatidylcholine ae C40:2 | HMDB13437 | na | C48H96NO7P |
| PC ae C40:3 | Phosphatidylcholine ae C40:3 | HMDB13445 | na | C48H92NO7P |
| PC ae C40:4 | Phosphatidylcholine ae C40:4 | HMDB13442 | na | C48H90NO7P |
| PC ae C40:5 | Phosphatidylcholine ae C40:5 | HMDB13444 | na | C48H88NO7P |
| PC ae C40:6 | Phosphatidylcholine ae C40:6 | HMDB13422 | na | C48H86NO7P |
| PC ae C42:0 | Phosphatidylcholine ae C42:0 | HMDB13443 | na | C50H102NO7P |
| PC ae C42:3 | Phosphatidylcholine ae C42:3 | HMDB13459 | na | C50H96NO7P |
| PC ae C44:3 | Phosphatidylcholine ae C44:3 | HMDB13449 | na | C52H100NO7P |
| PC ae C44:5 | Phosphatidylcholine ae C44:5 | HMDB13456 | na | C52H96NO7P |
| PC ae C44:6 | Phosphatidylcholine ae C44:6 | HMDB13457 | na | C52H94NO7P |
| SM C16:1 | Sphingomyelin C16:1 | HMDB06317 | na | C23H43NO4 |
| SM(OH) | Hydroxysphingomyelin C16:1 | HMDB13463 | na | C39H77N2O7P |
| C16:1 | | | | |
| SM(OH) | Hydroxysphingomyelin C22:2 | HMDB13467 | na | C45H87N2O7P |
| C22:2 | | | | |
| SM C18:0 | Sphingomyelin C18:0 | HMDB01348 | 58909- | C41H84N2O6P |
| | | | 84-5 | |
| SM C18:1 | Sphingomyelin C18:1 | HMDB12101 | 108392- | C41H81N2O6P |
| | | | 10-5 | |
| SM C20:2 | Sphingomyelin 20:2 | HMDB13465 | na | C43H83N2O6P |
| SM C22:3 | Sphingomyelin C22:3 | HMDB13468 | na | C45H85N2O6P |
| SM C24:1 | Sphingomyelin C24:1 | HMDB12107 | 94359- | C47H93N2O6P |
| | | | 1.3-4 | |
| lysoPC a | Lysophosphatidylcholine a C14:0 | HMDB10379 | 20559- | C22H46NO7P |
| C14:0 | | | 16-4 | |
| lysoPC a | Lysophosphatidylcholine a C16:0 | HMDB10382 | 17364- | C24H50NO7P |
| C16:0 | | | 16-8 | |
| LysoPC a | Lysophosphatidylcholine a C17:0 | HMDB12108 | 50930- | C25H52NO7P |
| C17:0 | | | 23-9 | |
| lysoPC | Lysophosphatidylcholine a C18:1 | HMDB02815 | 19420 | C26H52NO7P |
| a | | | 56-5 | |
| C18:1 | | | | |
| lysoPC a | Lysophosphatidylcholine a C18:2 | HMDB10386 | 22252- | C26H50NO7P |
| C18:2 | | | 07-9 | |
| lysoPC a | Lysophosphatidylcholine a C20:4 | HMDB10395 | 60701- | C28H50NO7P |
| C20:4 | | | 99-7 | |
| C0 | L-Carnitine (free carnitine) | HMDB00062 | 541-15-1 | C7H15NO3 |
| C3 | Propionylcarnitine | HMDB00824 | 20064- | C10H20NO4 |
| | | | 19-1 | |
| C3-DC (C4- | Hydroxybutyrylcarnitine | HMDB02095 | 910825- | C10H17NO6 |
| OH) | | | 21-7 | |
| C4 | Isobutyryl-L-carnitine | HMDB00736 | 25518- | C11H21NO4 |
| | | | 49-4 | |
| C5 | Isovalerylcarnitine | HMDB00688 | 31023- | C12H23NO4 |
| | | | 24-2 | |
| C5:1 | Tiglylcarnitine | HMDB02366 | 64681- | C12H21NO4 |
| | | | 36-3 | |
| C5-M-DC | Methylglutaryl-L-carnitine | HMDB00552 | 102673- | C12H25NO5 |
| | | | 95-0 | |
| C6:1 | Hexenoylcarnitine | HMDB13161 | na | C13H23NO4 |
| C8 | Octanoylcarnitine | HMDB0000791 | 25243- | C15H30NO4 |
| | | | 95-2 | |
| C10 | Decanoylcarnitine | HMDB00651 | 1492- | C17H33NO4 |
| | | | 27-9 | |
| C10:1 | Decenoylcarnitine | HMDB13205 | na | C17H31NO4 |
| C12-DC | Dodecanedioylcarnitine | HMDB13327 | na | C19H35NO6 |
| C14:1 | Tetradecenoylcarnitine | HMDB02014 | 835598- | C21H39NO4 |
| | | | 21-5 | |
| C14:2 | Tetradecadienylcarnitine | HMDB13331 | na | C21H37NO4 |
| C14:2-OH | Hydroxytetradecadienylcarnitine | HMDB240755 | na | C21H37NO5 |
| C16 | Hexadecanoylcarnitine | HMDB00222 | 2364- | C23H45NO4 |
| | | | 67-2 | |
| C16:2-OH | Hydroxyhexadecadienylcarnitine | HMDB13335 | na | C23H41NO5 |
| C18 | Stearoylcarnitine | HMDB00848 | 25597- | C25H50NO4 |
| | | | 09-5 | |
| C18:2 | Octadecadienylcarnitine | HMDB06461 | 85114- | C25H45NO4 |
| | | | 47-2 | |
| Arg | L-Arginine | HMDB00517 | 74-79-3 | C6H14N4O2 |
| Gln | L-Glutamine | HMDB00641 | 56-85-9 | C5H10N2O3 |
| Gly | L-Glycine | HMDB00123 | 56-40-6 | C2H5NO2 |
| Met | Methionine | HMDB00696 | 63-68-3 | C5H11NO2S |
| Orn | L-Ornitine | HMDB00214 | 3184- | C5H12N2O2 |
| | | | 13-2 | |
| Pro | L-Proline | HMDB00162 | 147-85-3 | C5H9NO2 |
| Ser | L-Serine | HMDB00187 | 56-45-1 | C3H7NO3 |
| Thr | L-Threonine | HMDB00167 | 72-19-5 | C4H9NO3 |
| Trp | L-Tryptophan | HMDB00929 | 73-22-3 | C11H12N2O2 |
| Tyr | L-Tyrosine | HMDB00158 | 60-18-4 | C9H11NO3 |

Abbreviations used in the table are explained as follows: HMDB—Human Metabolome Database (http://www.hmdb.ca) which provides annotation of chemical and biological parameters of a metabolite; CAS—Chemical Abstracts Service (http://www.cas.org) which provides annotation of chemical and physical parameters of a metabolite; na—not annotated, the “na” metabolite can be unequivocally measured but has not been described in the specific database.

The metabolites referred to herein are abbreviated using standard abbreviations well known in the art. Accordingly, “PC” abbreviates phosphatidylcholines, “LysoPC” abbreviates Lysophosphatidyl-choline, “SM” abbreviates sphingomyelins and “C0” abbreviates free carnitine. The term “Cx:y” is used to describe the total number of carbons (x) and the number of double bonds (y) of all chains. Substitutions of side chains with hydroxy-(OH) residue are indicated. Glycerophospholipids are distinguished with respect to the presence of ester (a) and ether (e) bonds in the glycerol moiety, where two letters (aa=diacyl, ae=acyl-alkyl) denote that the two glycerol positions are each bound to a fatty acid residue, while a single letter (a=acyl or e=alkyl) indicates the presence of a single fatty acid residue. For example “PC ae C34:1” denotes a glycerophosphatidylcholine with an acyl (a) and an ether (e) side chain, with 34 carbon atoms in both side chains and a single double bond in one of them. Amino acids are abbreviated in three letter code (e.g. Gln).

Further, the diagnostic approach according to the present invention involves use of a generalized linear model (GLM) based on the quantification of the at least two pairs, preferably at least three pairs, of metabolic biomarkers in a sample obtained from said subject. GLM is a statistical approach which is well established and widely used. The GLM generalizes linear regression by allowing the linear model to be related to the response variable via a link function and by allowing the magnitude of the variance of each measurement to be a function of its predicted value. The generalized linear models established by the present inventors allow the calculation of GLM-values characteristic for distinct endometriosis forms. The calculation can be performed for diagnosis of any form of endometriosis (all endometriosis), the diagnosis of a specific form like ovarian or peritoneal, and/or the diagnosis of mixed (multiple) forms like ovarian with coincidence of peritoneal and/or infiltrating.

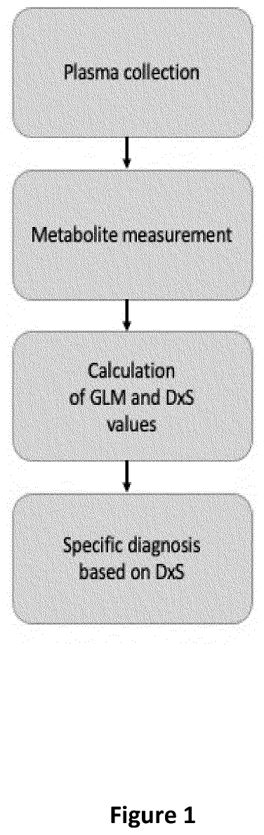

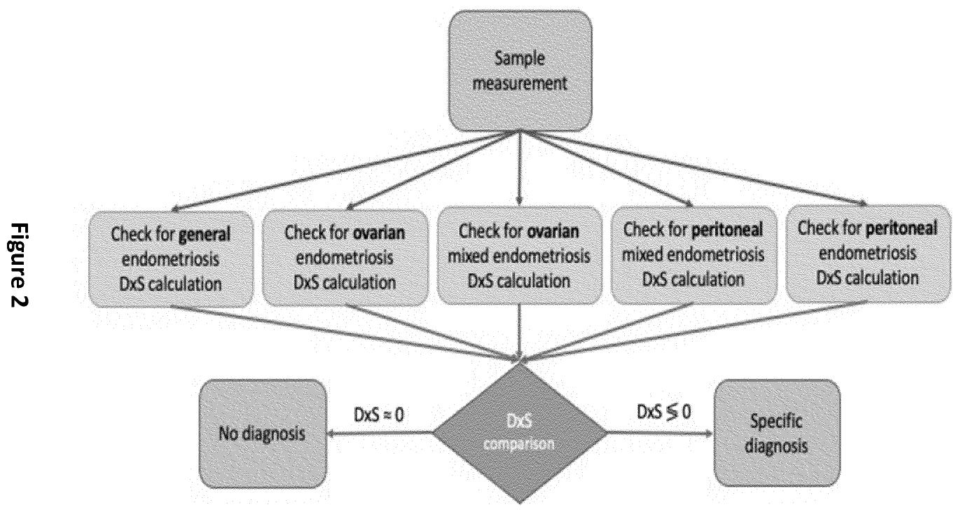

With the GLM-based diagnostic approach of the present invention it is thus not only made possible to determine from a single sample of a subject whether said subject is generally suffering from any form of endometriosis (all endometriosis), but also whether said subject is suffering from a specific form, like ovarian or peritoneal, or a mixed (multiple) form. The determination of the various forms can thereby be implemented as illustrated in FIGS. 1 and 2.

The present invention thus provides in a first aspect the use of a combination of at least two pairs, preferably at least three pairs, of metabolic biomarkers for the diagnosis of endometriosis and/or any sub-type thereof in a subject. More specifically, the present invention provides the use of a combination of at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH;
for the diagnosis of endometriosis and/or any sub-type thereof in a subject.

According to some embodiments, the present invention provides the use of a combination of at least two pairs, preferably at least three pairs, of metabolic biomarkers for diagnosing all endometriosis. More specifically, the present invention provides the use of a combination of at least two pairs, preferably at least three pairs, of metabolic biomarkers for diagnosing all endometriosis, wherein the at least two pairs, preferably at least three pairs, of metabolic biomarkers are selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; and Tyr and PC aa C42:4; C3-DC and C18; PC aa C42:1 and SM C22:3; and C6(C4:1-DC) and SM C16:1.

According to some embodiments, the present invention provides the use of a combination of at least two pairs, preferably at least three pairs, of metabolic biomarkers for diagnosing peritoneal endometriosis. More specifically, the present invention provides the use of a combination of at least two pairs, preferably at least three pairs, of metabolic biomarkers for diagnosing peritoneal endometriosis, wherein the at least two pairs, preferably at least three pairs, of metabolic biomarkers are selected from the group of pairs consisting of Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; and PC ae C34:1 and PC ae C42:0.

According to some embodiments, the present invention provides the use of a combination of at least two pairs, preferably at least three pairs, of metabolic biomarkers for diagnosing peritoneal mixed endometriosis. More specifically, the present invention provides the use of a combination of at least two pairs, preferably at least three pairs, of metabolic biomarkers for diagnosing peritoneal mixed endometriosis, wherein the at least two pairs, preferably at least three pairs, of metabolic biomarkers are selected from the group of pairs consisting of Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; SM C18:0 and C5; Gly and SM C24:1; PC aa C32:0 and PC aa C40:1; PC aa C36:4 and PC aa C38:0; Gly and PC aa C42:5; and C0 and SM(OH) C22:2.

According to some embodiments, the present invention provides the use of a combination of at least two pairs, preferably at least three pairs, of metabolic biomarkers for diagnosing ovarian endometriosis. More specifically, the present invention provides the use of a combination of at least two pairs, preferably at least three pairs, of metabolic biomarkers for diagnosing ovarian endometriosis, wherein the at least two pairs, preferably at least three pairs, of metabolic biomarkers are selected from the group of pairs consisting of PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C0 and C5-M-DC; C3 and PC ae 34:0.

According to some embodiments, the present invention provides the use of a combination of at least two pairs, preferably at least three pairs, of metabolic biomarkers for diagnosing ovarian mixed endometriosis. More specifically, the present invention provides the use of a combination of at least two pairs, preferably at least three pairs, of metabolic biomarkers for diagnosing ovarian mixed endometriosis, wherein the at least two pairs, preferably at least three pairs, of metabolic biomarkers are selected from the group of pairs consisting of C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; PC ae C44:3 and CPT I ratio; PC ae C34:0 and PC ae C40:3; C16:2-OH and SM C20:2; PC ae C44:6 and SM C22:3; and C10:1 and C14:2-OH.

According to some embodiments, the diagnosis involves use of a generalized linear model (GLM) based on the quantification of the at least two pairs, preferably at least three pairs, of metabolic biomarkers in a sample obtained from said subject.

The present invention provides in a further aspect an ex vivo method of diagnosing endometriosis and/or any subtype thereof in a subject comprising a) quantifying in a sample obtained from said of at least two pairs, preferably at least three pairs, of metabolic biomarkers, determining the ratio for each of the at least two pairs and b) obtaining a diagnostic score using a generalized linear model (GLM). More specifically, the present invention provides an ex vivo method of diagnosing endometriosis and/or any subtype thereof in a subject, the method comprising
a) quantifying in a sample obtained from said subject at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; PC ae C34:1 and PC ae C42:0; Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; LysoPC a C20:4 and PC aa C32:3; C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; Tyr and PC aa C42:4; C3-DC and C18; PC aa C42:1 and SM C22:3; Gly and SM C24:1; PC aa C32:0 and PC aa C40:1; PC aa C36:4 and PC aa C38:0; PC ae C44:3 and CPT I ratio; PC ae C34:0 and PC ae C40:3; C16:2-OH and SM C20:2; C6(C4:1-DC) and SM C16:1; Gly and PC aa C42:5; C0 and SM(OH) C22:2; PC ae C44:6 and SM C22:3; and C10:1 and C14:2-OH;
and b) obtaining a diagnostic score using a generalized linear model (GLM).

The method of the present invention may be performed to determined whether the subject is suffering from any type of endometriosis (all endometriosis), to determine whether the subject is suffering from a specific forms of endometriosis and/or to determine whether the subject is suffering from a mixed form of endometriosis. In other words, the method of the present invention may be performed to determine only one of any type of endometriosis (all endometriosis), a specific forms of endometriosis and a mixed form of endometriosis, or may be performed to determine two or more (such as all) of any type of endometriosis (all endometriosis), a specific form of endometriosis and a mixed form of endometriosis.

Thus, according to some embodiments, the method according to the present invention comprises determining whether the subject is suffering from any type of endometriosis comprising
A1) quantifying in a sample obtained from said subject at least two pairs, preferably at least three pairs, of metabolic biomarkers selected from the group of pairs consisting of LysoPC a C17:0 and SM(OH) C16:1; Arg and PC ae C36:0; PC ae C38:0 and PC ae C40:0; LysoPC a C16:0 and SM C18:1; Thr and PC aa C34:3; C18 and LysoPC a C14:0; Ser and PC ae C44:3; Trp and PC ae C38:3; C8 and PC ae C30:0; Thr and PC ae C36:5; C10 and PC ae C38:6; Arg and PC aa C36:6; and Tyr and PC aa C42:4; C3-DC and C18; PC aa C42:1 and SM C22:3; and C6(C4:1-DC) and SM C16:1; and
B1) obtaining a diagnostic score using a generalized linear model (GLM).

According to some embodiments, the method according to the present invention (further) comprises determining whether the subject is suffering from peritoneal endometriosis comprising
A2) quantifying in a sample obtained from said subject at least two pairs, preferably at least three pairs, of metabolic biomarkers selected from the group of pairs consisting of Thr and SM(OH) C22:2; LysoPC a C16:0 and SM(OH) C16:1; PC aa C32:0 and SM C18:0; PC aa C32:0 and PC aa C38:3; C6:1 and Pro; Arg and PC ae C34:0; C6:1 and LysoPC a C20:4; C5-M-DC and PC aa C42:5; LysoPC a C18:2 and PC ae C40:6; LysoPC a C18:2 and PC ae C40:4; PC ae C40:6 and CPT I ratio; LysoPC a C17:0 and SM C18:0; C4 and PC ae C30:2; Arg and PC ae C34:0; and PC ae C34:1 and PC ae C42:0; and
B2) obtaining a diagnostic score using a generalized linear model (GLM).

According to some embodiments, the method according to the present invention (further) comprises determining whether the subject is suffering from peritoneal mixed endometriosis comprising
A3) quantifying in a sample obtained from said subject at least two pairs, preferably at least three pairs, of metabolic biomarkers selected from the group of pairs consisting of Orn and PC ae C38:0; C4 and PC aa C38:4; Tyr and PC aa C42:2; Arg and PC aa C36:6; C5 and LysoPC a C17:0; C5 and Arg; C0 and Gly; Ser and SM(OH) C16:1; C3 and PC ae C40:5; Pro and PC ae C34:0; C4 and Ser; C4 and PC ae C40:3; PC ae C42:3 and SM(OH) C16:1; Tyr and PC ae C38:0; Gly and SM C24:1; PC aa C32:0 and PC aa C40:1; PC aa C36:4 and PC aa C38:0; Gly and PC aa C42:5; and C0 and SM(OH) C22:2; and
B3) performing generalized linear modelling (GLM).

According to some embodiments, the method according to the present invention (further) comprises determining whether the subject is suffering from ovarian endometriosis comprising
A4) quantifying in a sample obtained from said subject at least two pairs, preferably at least three pairs, of metabolic biomarkers selected from the group of pairs consisting of PC aa C36:3 and PC ae C40:5; LysoPC a C14:0 and PC aa C28:1; Met and PC aa C36:3; PC aa C38:0 and PC ae C36:1; Thr and SM (OH) C22:1; PC aa C28:1 and PC ae C34:3; C18:2 and PC ae C34:3; C3 and PC ae C34:1; Gly and PC ae C36:1; C10:1 and PC aa C36:1; PC ae C38:3 and SM C18:1; C12-DC and C14:2; PC aa C38:3 and PC ae C44:5; C4 and C5:1; LysoPC a C20:4 and PC ae C32:1; and LysoPC a C20:4 and PC aa C32:3; and
B4) performing generalized linear modelling (GLM).

According to some embodiments, the method according to the present invention (further) comprises determining whether the subject is suffering from ovarian mixed endometriosis comprising
A5) quantifying in a sample obtained from said subject at least two pairs, preferably at least three pairs, of metabolic biomarkers selected from the group of pairs consisting of C10 and PC aa C36:6; PC ae C42:3 and SM(OH) C16:1; Pro and PC ae C34:0; C6:1 and LysoPC a C20:4; LysoPC a C20:4 and PC ae C40:2; Ser and PC aa C38:3; C10 and LysoPC a C18:1; LysoPC a C24:0 and PC ae C42:3; LysoPC a C18:1 and PC aa C36:1; Gly and PC ae C34:1; Gln and PC ae C30:2; LysoPC a C24:0 and PC ae C42:3; C10:1 and LysoPC a C24:0; PC ae C44:3 and CPT I ratio; PC ae C34:0 and PC ae C40:3; C16:2-OH and SM C20:2; PC ae C44:6 and SM C22:3; and C10:1 and C14:2-OH; and
B5) performing generalized linear modelling (GLM).

The generalized linear model(s) used according to the present invention may comprise determining the ratio of the concentrations for each of the at least two pairs, preferably at least three pairs, of metabolic biomarkers; and calculating the sum of the obtained ratios (value for case). The calculated sum of the obtained ratios (value for case) may then be compared to a predetermined reference value established from healthy subjects (value for control) applying the same GLM on the respective metabolites quantified in samples of said healthy subjects.

Specifically, a diagnostic score (DxS) can then be calculated by forming the quotient between a predetermined reference value obtained from healthy subjects (value for control) and the sum of the obtained ratios (value for case)

$$DxS = log 2 ( \frac{predetermined reference value ( value for control )}{sum of the obtained ratios ( value for case )} )$$

The DxS is enabling mathematic values obtained from calculations of metabolite ratios according to models (GLMs in the diagnostics). DxS values in the range of 0±0.03 are not facilitating diagnosis of specific indication of endometriosis type and other models have to be taken into the consideration as described in FIGS. 1 and 2.

“Healthy subjects” in accordance with the present invention are subjects that do not have endometriosis. Accordingly, it will be appreciated that the term “healthy subject”, in accordance with the present invention, does not require an overall healthy subject. Instead, a healthy subject in accordance with the present invention is a person not having endometriosis. Whether a subject has endometriosis can be ascertained by the presence of a plurality, such as e.g. at least three, more preferably at least four, such as at least five and most preferably all of the unspecific diagnostic parameters including: normal fertility, no pelvic pain or no pain in lower abdomen before menstruation, no pain with bowel movements, lack of inflammatory biomarkers, lack of extra menstrual bleeding. However, as final and dependable diagnosis of endometriosis depends on laparoscopic examination, which is an invasive operative procedure, it is preferred that the healthy subjects are subjects for which the absence of endometriosis has been confirmed by laparoscopic examination.

For example, samples may be taken from a sufficiently large group of healthy subjects, such as for example at least 10, more preferably at least 75 and most preferably at least 100 healthy subjects. The metabolite values obtained from this group, which are also referred to herein as reference values, are then correlated with the absence of endometriosis. It will be appreciated by the skilled person that determining these reference values in healthy subjects may be carried out prior to performing the present invention, such that the determined values may be used as a reference at later times whenever a sample is analysed in accordance with the present invention; or may be determined in parallel each time a sample is analysed in accordance with the present invention. Such reference values may also be determined only once and stored as a standard for all future tests.

Preferably, the reference values are derived from a population having the same racial background as the women to be diagnosed. For example, when employing the present invention in e.g. caucasian women, the reference values should be obtained from healthy caucasian subjects.

For example, using a group of caucasian (i.e. Slovenian and Austrian) females as shown in the appended examples, reference values where determined as shown in Tables 5, 8, 11, 14 and 17 below.

Accordingly, when employing the method of the present invention in a group of caucasian females, the above defined reference values for healthy subjects may for example be relied upon.

Generally, an indication of endometriosis or any of its sub-types is given when the diagnostic score is different from zero (“0”). In other words, if the diagnostic score has a positive or negative value, then the subject can be diagnosed as having endometriosis or the sub-type investigate. Conversely, if the diagnostic score is zero (“0”), then the subject is not suffering from endometriosis or the sub-type investigate.

According to some embodiments, the method according to the present invention comprises determining whether the subject is suffering from any type of endometriosis comprising any one of the following procedures (1) to (15):
(1) quantifying in a sample obtained from said subject the metabolites LysoPC a C17:0, SM(OH) C16:1, Arg, PC ae C36:0, PC ae C38:0 and PC ae C40:0; and performing GLM using the model formula LysoPC a C17:0_div_by_SM(OH) C16:1+Arg_div_by_PC ae C36:0+PC ae C38:0_div_by_PC ae C40:0;
(2) quantifying in a sample obtained from said subject the metabolic biomarkers Arg, PC ae C36:0, LysoPC a C16:0, SM C18:1, PC ae C38:0 and PC ae C40:0; and performing GLM using the model formula Arg_div_by_PC ae C36:0+lysoPC a C16:0_div_by_SM C18:1+PC ae C38:0_div_by_PC ae C40:0;
(3) quantifying in a sample obtained from said subject the metabolic biomarkers Thr, PC aa C34:3, LysoPC a C17:0, SM(OH) C16:1, Arg and PC ae C36:0; and performing GLM using the model formula Thr_div_by_PC aa C34:3+LysoPC a C17:0_div_by_SM(OH) C16:1+Arg_div_by_PC ae C36:0;
(4) quantifying in a sample obtained from said subject the metabolic biomarkers Thr, PC aa C34:3, Arg, PC ae C36:0, LysoPC a C16:0 and SM C18:1; and performing GLM using the model formula Thr_div_by_PC aa C34:3+Arg_div_by_PC ae C36:0+lysoPC a C16:0_div_by_SM C18:1;
(5) quantifying in a sample obtained from said subject the metabolic biomarkers Arg, PC aa C36:6, LysoPC a C17:0, SM(OH) C16:1, and PC ae C36:0; and performing GLM using the model formula Arg_div_by_PC aa C36:6+LysoPC a C17:0_div_by_SM(OH) C16:1+Arg_div_by_PC ae C36:0;
(6) quantifying in a sample obtained from said subject the metabolic biomarkers LysoPC a C17:0, SM(OH) C16:1, Arg, PC ae C36:0, C18 and LysoPC a C14:0; and performing GLM using the model formula LysoPC a C17:0_div_by_SM(OH) C16:1+Arg_div_by_PC ae C36:0+C18_div_by_LysoPC a C14:0;
(7) quantifying in a sample obtained from said subject the metabolic biomarkers LysoPC a C16:0, SM C18:1, PC ae C38:0, PC ae C40:0, Ser and PC ae C44:3; and performing GLM using the model formula LysoPC a C16:0_div_by_SM C18:1+PC ae C38:0_div_by_PC ae C40:0+Ser_div_by_PC ae C44:3;
(8) quantifying in a sample obtained from said subject the metabolic biomarkers LysoPC a C17:0, SM(OH) C16:1, Arg, PC ae C36:0, Trp, PC ae C38:3; and performing GLM using the model formula LysoPC a C17:0_div_by_SM(OH) C16:1+Arg_div_by_PC ae C36:0+Trp_div_by_PC ae C38:3;
(9) quantifying in a sample obtained from said subject the metabolic biomarkers Arg, PC ae C36:0, LysoPC a C16:0, SM C18:1, C8 and PC ae C30:0; and performing GLM using the model formula Arg_div_by_PC ae C36:0+LysoPC a C16:0_div_by_SM C18:1+C8_div_by_PC ae C30:0;
(10) quantifying in a sample obtained from said subject the metabolic biomarkers Thr, PC ae C36:5, Arg, PC ae C36:0, LysoPC a C16:0 and SM C18:1; and performing GLM using the model formula Thr_div_by_PC ae C36:5+Arg_div_by_PC ae C36:0+LysoPC a C16:0_div_by_SM C18:1;
(11) quantifying in a sample obtained from said subject the metabolite Arg, PC ae C36:0, C18, LysoPC a C14:0, LysoPC a C16:0 and SM C18:1; and performing GLM using the model formula Arg_div_by_PC ae C36:0+C18_div_by_LysoPC a C14:0+LysoPC a C16:0_div_by_SM C18:1;
(12) quantifying in a sample obtained from said subject the metabolic biomarkers Arg, PC ae C36:0, C10, PC ae C38:6, LysoPC a C16:0 and SM C18:1; and performing GLM using the model formula Arg_div_by_PC ae C36:0+C10_div_by_PC ae C38:6+LysoPC a C16:0_div_by_SM C18:1;
(13) quantifying in a sample obtained from said subject the metabolic biomarkers Arg, PC aa C36:6, PC ae C36:0, LysoPC a C16:0 and SM C18:1; and performing GLM using the model formula Arg_div_by_PC aa C36:6+Arg_div_by_PC ae C36:0+lysoPC a C16:0_div_by_SM C18:1;
(14) quantifying in a sample obtained from said subject the metabolic biomarkers Tyr, PC aa C42:4, C3-DC, C18, PC aa C42:1 and SM C22:3; and performing GLM using the model formula Tyr_div_by_PC aa C42:4+C3-DC_div_by_C18+PC aa C42:1_div_by_SM C22:3;
(15) quantifying in a sample obtained from said subject the metabolic biomarkers C3-DC, C18, PC aa C42:1, SM C22:3, C6(C4:1-DC) and SM C16:1; and performing GLM using the model formula C3-DC_div_by_C18+PC aa C42:1_div_by_SM C22:3+C6 (C4:1-DC)_div_by_SM C16:1.

According to some embodiments, the method according to the present invention comprises determining whether the subject is suffering from peritoneal endometriosis comprising any one of the following procedures (1) to (7):
(1) quantifying in a sample obtained from said subject the metabolic biomarkers LysoPC a C16:0, SM(OH) C16:1, PC aa C32:0, SM C18:0, PC aa C32:0 and PC aa C38:3; and performing GLM using the model formula LysoPC a C16:0_div_by_SM(OH) C16:1+PC aa C32:0_div_by_SM C18:0+PC aa C32:0_div_by_PC aa C38:3;
(2) quantifying in a sample obtained from said subject the metabolic biomarkers LysoPC a C16:0, SM(OH) C16:1, PC aa C32:0, SM C18:0, Arg and PC ae C34:0; and performing GLM using the model formula LysoPC a C16:0_div_by_SM(OH) C16:1+PC aa C32:0_div_by_SM C18:0+Arg_div_by_PC ae C34:0;
(3) quantifying in a sample obtained from said subject the metabolic biomarkers C5-M-DC, PC aa C42:5, Arg, PC ae C34:0, LysoPC a C18:2 and PC ae C40:6; and performing GLM using the model formula C5-M-DC_div_by_PC aa C42:5+Arg_div_by_PC ae C34:0+LysoPC a C18:2_div_by_PC ae C40:6;
(4) quantifying in a sample obtained from said subject the metabolic biomarkers LysoPC a C18:2, PC ae C40:4, PC ae C40:6, CPT I ratio, LysoPC a C17:0 and SM C18:0; and performing GLM using the model formula LysoPC a C18:2_div_by_PC ae C40:4+PC ae C40:6_div_by_CPT I ratio+lysoPC a C17:0_div_by_SM C18:0;
(5) quantifying in a sample obtained from said subject the metabolic biomarkers C4, PC ae C30:2, Arg, PC ae C34:0, LysoPC a C18:2 and PC ae C40:6; and performing GLM using the model formula C4_div_by_PC ae C30:2+Arg_div_by_PC ae C34:0+LysoPC a C18:2_div_by_PC ae C40:6;
(6) quantifying in a sample obtained from said subject the metabolic biomarkers PC ae C40:6, CPT I ratio, C4, PC ae C30:2, LysoPC a C18:2 and PC ae C40:6; and performing GLM using the model formula PC ae C40:6_div_by_CPT I ratio+C4_div_by_PC ae C30:2+LysoPC a C18:2_div_by_PC ae C40:6;
(7) quantifying in a sample obtained from said subject the metabolic biomarkers LysoPC a C18:2, PC ae C40:4, Arg, PC ae C34:0, PC ae C34:1 and PC ae C42:0; and performing GLM using the model formula LysoPC a C18:2_div_by_PC ae C40:4+Arg_div_by_PC ae C34:0+PC ae C34:1_div_by_PC ae C42:0.

According to some embodiments, the method according to the present invention comprises determining whether the subject is suffering from peritoneal mixed endometriosis comprising any one of the following procedures (1) to (16):
(1) quantifying in a sample obtained from said subject the metabolic biomarkers Orn, PC ae C38:0, C4, PC aa C38:4, Tyr and PC aa C42:2; and performing GLM using the model formula Orn_div_by_PC ae C38:0+C4_div_by_PC aa C38:4+Tyr_div_by_PC aa C42:2;
(2) quantifying in a sample obtained from said subject the metabolic biomarkers Arg, PC aa C36:6, C5 and LysoPC a C17:0; and performing GLM using the model formula Arg_div_by_PC aa C36:6+C5_div_by_lysoPC a C17:0+C5_div_by_Arg;
(3) quantifying in a sample obtained from said subject the metabolic biomarkers Orn, PC ae C38:0, C5, LysoPC a C17:0 and Arg; and performing GLM using the model formula Orn_div_by_PC ae C38:0+C5_div_by_lysoPC a C17:0+C5_div_by_Arg;
(4) quantifying in a sample obtained from said subject the metabolic biomarkers C0, Gly, Orn, PC ae C38:0, Tyr and PC aa C42:2; and performing GLM using the model formula C0_div_by_Gly+Orn_div_by_PC ae C38:0+Tyr_div_by_PC aa C42:2;
(5) quantifying in a sample obtained from said subject the metabolic biomarkers SM C18:0, C5, LysoPC a C17:0 and Arg; and performing GLM using the model formula SM C18:0+C5_div_by_lysoPC a C17:0+C5_div_by_Arg;
(6) quantifying in a sample obtained from said subject the metabolic biomarkers C5, LysoPC a C17:0, Arg, Ser and SM(OH) C16:1; and performing GLM using the model formula C5_div_by_lysoPC a C17:0+C5_div_by_Arg+Ser_div_by_SM(OH) C16:1;
(7) quantifying in a sample obtained from said subject the metabolic biomarkers SM C18:0, C0, Gly, Tyr and PC aa C42:2; and performing GLM using the model formula SM C18:0+C0_div_by_Gly+Tyr_div_by_PC aa C42:2;
(8) quantifying in a sample obtained from said subject the metabolic biomarkers Orn, PC ae C38:0, C3, PC ae C40:5, Tyr and PC aa C42:2; and performing GLM using the model formula Orn_div_by_PC ae C38:0+C3_div_by_PC ae C40:5+Tyr_div_by_PC aa C42:2;
(9) quantifying in a sample obtained from said subject the metabolic biomarkers Pro, PC ae C34:0, Orn, PC ae C38:0, Tyr and PC aa C42:2; and performing GLM using the model formula Pro_div_by_PC ae C34:0+Orn_div_by_PC ae C38:0+Tyr_div_by_PC aa C42:2;
(10) quantifying in a sample obtained from said subject the metabolic biomarkers C4, Ser, Orn, PC ae C38:0, Tyr and PC aa C42:2; and performing GLM using the model formula C4_div_by_Ser+Orn_div_by_PC ae C38:0+Tyr_div_by_PC aa C42:2;
(11) quantifying in a sample obtained from said subject the metabolic biomarkers SM C18:0, Orn, PC ae C38:0, Tyr and PC aa C42:2; and performing GLM using the model formula SM C18:0+Orn_div_by_PC ae C38:0+Tyr_div_by_PC aa C42:2;
(12) quantifying in a sample obtained from said subject the metabolic biomarkers Orn, PC ae C38:0, C4, PC ae C40:3, Tyr and PC aa C42:2; and performing GLM using the model formula Orn_div_by_PC ae C38:0+C4_div_by_PC ae C40:3+Tyr_div_by_PC aa C42:2;
(13) quantifying in a sample obtained from said subject the metabolic biomarkers Orn, PC ae C38:0, PC ae C42:3, SM(OH) C16:1, Tyr and PC aa C42:2; and performing GLM using the model formula Orn_div_by_PC ae C38:0+PC ae C42:3_div_by_SM(OH) C16:1+Tyr_div_by_PC aa C42:2;
(14) quantifying in a sample obtained from said subject the metabolic biomarkers Orn, PC ae C38:0, Tyr, PC ae C38:0 and PC aa C42:2; and performing GLM using the model formula Orn_div_by_PC ae C38:0+Tyr_div_by_PC ae C38:0+Tyr_div_by_PC aa C42:2;
(15) quantifying in a sample obtained from said subject the metabolic biomarkers Gly, SM C24:1, PC aa C32:0, PC aa C40:1, PC aa C36:4 and PC aa C38:0; and performing GLM using the model formula Gly_div_by_SM C24:1+PC aa C32:0_div_by_PC aa C40:1+PC aa C36:4_div_by_PC aa C38:0;
(16) quantifying in a sample obtained from said subject the metabolic biomarkers Gly, PC aa C42:5, PC aa C36:4, PC aa C38:0SM, C0 and SM(OH) C22:2; and performing GLM using the model formula Gly_div_by_PC aa C42:5+PC aa C36:4_div_by_PC aa C38:0+C0_div_by_SM(OH) C22:2.

According to some embodiments, the method according to the present invention comprises determining whether the subject is suffering from ovarian endometriosis comprising any one of the following procedures (1) to (14):
(1) quantifying in a sample obtained from said subject the metabolic biomarkers PC aa C36:3, PC ae C40:5, LysoPC a C14:0, PC aa C28:1 and Met; and performing GLM using the model formula PC aa C36:3_div_by_PC ae C40:5+LysoPC a C14:0_div_by_PC aa C28:1+Met_div_by_PC aa C36:3;
(2) quantifying in a sample obtained from said subject the metabolic biomarkers PC aa C38:0, PC ae C36:1, Thr, SM (OH) C22:1, LysoPC a C14:0 and PC aa C28:1; and performing GLM using the model formula PC aa C38:0_div_by_PC ae C36:1+Thr_div_by_SM (OH) C22:1+lysoPC a C14:0_div_by_PC aa C28:1;
(3) quantifying in a sample obtained from said subject the metabolic biomarkers Thr, SM (OH) C22:1, PC aa C28:1, PC ae C34:3, C18:2 and PC ae C34:3; and performing GLM using the model formula Thr_div_by_SM (OH) C22:1+PC aa C28:1_div_by_PC ae C34:3+C18:2_div_by_PC ae C34:3;
(4) quantifying in a sample obtained from said subject the metabolic biomarkers PC aa C36:3, PC ae C40:5, C3, PC ae C34:1, Met and PC aa C36:3; and performing GLM using the model formula PC aa C36:3_div_by_PC ae C40:5+C3_div_by_PC ae C34:1+Met_div_by_PC aa C36:3;
(5) quantifying in a sample obtained from said subject the metabolic biomarkers PC aa C36:3, PC ae C40:5, PC aa C28:1, PC ae C34:3, Gly and PC ae C36:1; and performing GLM using the model formula PC aa C36:3_div_by_PC ae C40:5+PC aa C28:1_div_by_PC ae C34:3+Gly_div_by_PC ae C36:1;
(6) quantifying in a sample obtained from said subject the metabolic biomarkers PC aa C38:0, PC ae C36:1, C18:2, PC ae C34:3, Met and PC aa C36:3; and performing GLM using the model formula PC aa C38:0_div_by_PC ae C36:1+C18:2_div_by_PC ae C34:3+Met_div_by_PC aa C36:3;
(7) quantifying in a sample obtained from said subject the metabolic biomarkers PC aa C38:0, PC ae C36:1, C10:1, PC aa C36:1, PC ae C38:3 and SM C18:1; and performing GLM using the model formula PC aa C38:0_div_by_PC ae C36:1+C10:1_div_by_PC aa C36:1+PC ae C38:3_div_by_SM C18:1;
(8) quantifying in a sample obtained from said subject the metabolic biomarkers PC aa C38:0, PC ae C36:1, Gly, C3 and PC ae C34:1; and performing GLM using the model formula PC aa C38:0_div_by_PC ae C36:1+Gly_div_by_PC ae C36:1+C3_div_by_PC ae C34:1;
(9) quantifying in a sample obtained from said subject the metabolic biomarkers PC aa C38:0, PC ae C36:1, C12-DC, C14:2, PC ae C38:3 and SM C18:1; and performing GLM using the model formula PC aa C38:0_div_by_PC ae C36:1+C12-DC_div_by_C14:2+PC ae C38:3_div_by_SM C18:1;
(10) quantifying in a sample obtained from said subject the metabolic biomarkers PC aa C36:3, PC ae C40:5, PC aa C38:3, PC ae C44:5 and Met; and performing GLM using the model formula PC aa C36:3_div_by_PC ae C40:5+PC aa C38:3_div_by_PC ae C44:5+Met_div_by_PC aa C36:3;
(11) quantifying in a sample obtained from said subject the metabolic biomarkers PC aa C38:0, PC ae C36:1, PC ae C38:3, SM C18:1, Met and PC aa C36:3; and performing GLM using the model formula PC aa C38:0_div_by_PC ae C36:1+PC ae C38:3_div_by_SM C18:1+Met_div_by_PC aa C36:3;
(12) quantifying in a sample obtained from said subject the metabolic biomarkers PC aa C28:1, PC ae C34:3, C18:2, PC ae C34:3, C4 and C5:1; and performing GLM using the model formula PC aa C28:1_div_by_PC ae C34:3+C18:2_div_by_PC ae C34:3+C4_div_by_C5:1;
(13) quantifying in a sample obtained from said subject the metabolic biomarkers PC aa C36:3, PC ae C40:5, LysoPC a C20:4, PC ae C32:1 and Met; and performing GLM using the model formula PC aa C36:3_div_by_PC ae C40:5+lysoPC a C20:4_div_by_PC ae C32:1+Met_div_by_PC aa C36:3;
(14) quantifying in a sample obtained from said subject the metabolic biomarkers PC aa C36:3, PC ae C40:5, LysoPC a C20:4, PC aa C32:3 and Met; and performing GLM using the model formula PC aa C36:3_div_by_PC ae C40:5+lysoPC a C20:4_div_by_PC aa C32:3+Met_div_by_PC aa C36:3.

According to some embodiments, the method according to the present invention comprises determining whether the subject is suffering from ovarian mixed endometriosis comprising any one of the following procedures (1) to (13):
(1) quantifying in a sample obtained from said subject the metabolic biomarkers C10, PC aa C36:6, Pro, PC ae C34:0, PC ae C42:3 and SM(OH) C16:1; and performing GLM using the model formula C10_div_by_PC aa C36:6+Pro_div_by_PC ae C34:0+PC ae C42:3_div_by_SM(OH) C16:1;
(2) quantifying in a sample obtained from said subject the metabolic biomarkers C10, PC aa C36:6, PC ae C42:3, SM(OH) C16:1, C6:1 and LysoPC a C20:4; and performing GLM using the model formula C10_div_by_PC aa C36:6+PC ae C42:3_div_by_SM(OH) C16:1+C6:1_div_by_lysoPC a C20:4;
(3) quantifying in a sample obtained from said subject the metabolic biomarkers C10, PC aa C36:6, PC ae C42:3, SM(OH) C16:1, LysoPC a C20:4 and PC ae C40:2; and performing GLM using the model formula C10_div_by_PC aa C36:6+PC ae C42:3_div_by_SM(OH) C16:1+lysoPC a C20:4_div_by_PC ae C40:2;
(4) quantifying in a sample obtained from said subject the metabolic biomarkers Ser, PC aa C38:3, C10, PC aa C36:6, PC ae C42:3 and SM(OH) C16:1; and performing GLM using the model formula Ser_div_by_PC aa C38:3+C10_div_by_PC aa C36:6+PC ae C42:3_div_by_SM(OH) C16:1;
(5) quantifying in a sample obtained from said subject the metabolic biomarkers C10, LysoPC a C18:1, PC aa C36:6, PC ae C42:3 and SM(OH) C16:1; and performing GLM using the model formula C10_div_by_lysoPC a C18:1+C10_div_by_PC aa C36:6+PC ae C42:3_div_by_SM(OH) C16:1;
(6) quantifying in a sample obtained from said subject the metabolic biomarkers C10, PC aa C36:6, LysoPC a C24:0, PC ae C42:3, PC ae C42:3 and SM(OH) C16:1; and performing GLM using the model formula C10_div_by_PC aa C36:6+LysoPC a C24:0_div_by_PC ae C42:3+PC ae C42:3_div_by_SM(OH) C16:1;
(7) quantifying in a sample obtained from said subject the metabolic biomarkers C10, PC aa C36:6, LysoPC a C18:1, PC aa C36:1, PC ae C42:3 and SM(OH) C16:1; and performing GLM using the model formula C10_div_by_PC aa C36:6+LysoPC a C18:1_div_by_PC aa C36:1+PC ae C42:3_div_by_SM(OH) C16:1;
(8) quantifying in a sample obtained from said subject the metabolic biomarkers C10, PC aa C36:6, +Gly, PC ae C34:1, PC ae C42:3 and SM(OH) C16:1; and performing GLM using the model formula C10_div_by_PC aa C36:6+Gly_div_by_PC ae C34:1+PC ae C42:3_div_by_SM(OH) C16:1;
(9) quantifying in a sample obtained from said subject the metabolic biomarkers Gln, PC ae C30:2, C10, PC aa C36:6, PC ae C42:3 and SM(OH) C16:1; and performing GLM using the model formula Gln_div_by_PC ae C30:2+C10_div_by_PC aa C36:6+PC ae C42:3_div_by_SM(OH) C16:1;
(10) quantifying in a sample obtained from said subject the metabolic biomarkers Pro, PC ae C34:0, LysoPC a C24:0, PC ae C42:3, PC ae C42:3 and SM(OH) C16:1; and performing GLM using the model formula Pro_div_by_PC ae C34:0+lysoPC a C24:0_div_by_PC ae C42:3+PC ae C42:3_div_by_SM(OH) C16:1;
(11) quantifying in a sample obtained from said subject the metabolic biomarkers C10:1, LysoPC a C24:0, LysoPC a C24:0, PC ae C42:3 and SM(OH) C16:1; and performing GLM using the model formula C10:1_div_by_lysoPC a C24:0+lysoPC a C24:0_div_by_PC ae C42:3+PC ae C42:3_div_by_SM(OH) C16:1;
(12) quantifying in a sample obtained from said subject the metabolic biomarkers PC ae C44:3, CPT I ratio, PC ae C34:0, PC ae C40:3, C16:2-OH and SM C20:2; and performing GLM using the model formula PC ae C44:3_div_by_CPT.I.ratio+PC ae C34:0_div_by_PC ae C40:3+C16:2-OH_div_by_SM C20:2;
(13) quantifying in a sample obtained from said subject the metabolic biomarkers PC ae C44:6, SM C22:3, PC ae C34:0, PC ae C40:3, C10:1 and C14:2-OH; and performing GLM using the model formula PC ae C44:6_div_by_SM C22:3+PC ae C34:0_div_by_PC ae C40:3+C10:1_div_by_C14:2-OH.

Means and methods for quantifying (i.e. determining the concentration) of metabolites in samples, such as e.g. in blood, are well known in the art. Preferably, quantifying the metabolic biomarkers includes measuring the absolute concentration of each of the biomarkers in the sample obtained from said subject.

Suitably, the metabolic biomarkers are to be quantified with mass spectrometry to ensure specificity of metabolite identification, quantification of metabolites and multiplexing. Thus, according to some embodiments, the concentrations of the metabolic biomarkers are determined by mass spectrometry.

Mass spectrometry and its use for determining the concentration of metabolites in a sample is well known in the art and has been described for example in (45 and 46). Mass spectrometry includes, for example, flow-injection analysis mass spectrometry (FIA-MS), tandem mass spectrometry, matrix assisted laser desorption ionization (MALDI) time-of-flight (TOF) mass spectrometry, MALDI-TOF-TOF mass spectrometry, MALDI Quadrupole-time-of-flight (Q-TOF) mass spectrometry, electrospray ionization (ESI)-TOF mass spectrometry, ESI-Q-TOF, ESI-TOF-TOF, ESI-ion trap mass spectrometry, ESI Triple quadrupole mass spectrometry, ESI Fourier Transform mass spectrometry (FTMS), MALDI-FTMS, MALDI-lon Trap-TOF, and ESI-Ion Trap TOF. At its most basic level, mass spectrometry involves ionizing a molecule and then measuring the mass of the resulting ions. Since molecules ionize in a way that is well known, the molecular weight of the molecule can be accurately determined from the mass of the ions. In addition, by a comparison of data obtained from internal standards, a quantification of molecules of interest is possible, as detailed herein below.

According to some embodiments, the mass spectrometry is selected from flow-injection analysis mass spectrometry (FIA-MS), liquid chromatography mass spectrometry (LC-MS or HPLC-MS) and tandem mass spectrometry (MS-MS).

The sample to be analysed may be any sample allowing the quantification of the metabolites. Non-limiting examples of suitable samples include blood, serum, plasma, saliva, urine, cerebrospinal fluid, condensates from respiratory air, tears, mucosal tissue, mucus, vaginal tissue, endometrium, eutopic endometrium, skin, hair or hair follicle, of which blood, serum and plasma are preferred.

According to some embodiments, the sample is selected from blood, serum and plasma.

According to some embodiments, the sample is plasma.

According to some embodiments, the subject is suspected to suffer from endometriosis or to have a predisposition therefore.

According to some embodiments, the subject is a human subject, and preferably a human female.

According to some embodiments, the human subject, preferably human female, is of Caucasian race.

### Certain Definitions

The expression “AUC” as used herein means “area under the curve” and describes the quality of diagnostic model. The worst value is 0.5, the theoretically best 1.0 (41).

The expression “GLM” as used herein means generalized linear model (42).

The expression “CPT I ratio” as used herein is a ratio of (C18AC+C16AC)/C0, i.e. (octadecanoylcarnitine+hexadecanoylcarnitine)/free carnitine. It describes efficiency of import of metabolites to mitochondria (38).

“Variance” is the expectation of the squared deviation of a random variable from its mean.

$$variance {σ}^{2} = \frac{{\sum }_{i = 1}^{n} {( {x}_{i} - \tilde{x} )}^{2}}{n}$$

where:
xᵢ=the i^{th} data point
x̅=the mean of all data points
n=the number of data points

“R_{γ}²” describes explained x-variation. Should be above 0.75 and never negative.

“Rₓ²” describes explained y-variation. Should be above 0.75 and never negative.

“Qₓ²” describes predicted variation. Should be above 0.4, never 1.0 and never negative.

“RMSE” means “root mean square error” of estimations and is an accuracy of the model. Should be below 0.25

“P_{R}²”— R² parameter after permutation testing of the sample grouping (2000 times). Has to be below 0.05 to produce valid PLS-DA.

“P_{Q}²”—Q² parameter after permutation testing of the sample grouping (²000 times). Has to be below 0.05 to produce a valid PLS-DA.

“Fold change” is expressed as log 2 value to enable linear comparison (39, 40).

The expression “_div_by_” as used herein corresponds to the division of concentration of two metabolites.

Having generally described this invention, a further understanding can be obtained by reference to certain specific examples, which are provided herein for purposes of illustration only, and are not intended to be limiting unless otherwise specified.

### EXAMPLES

### Description of Study and its Replication

For the discovery and replication studies we ensured that controls and cases are matched for age and BMI as far as the clinical setting allows. Patients with other comorbidities were excluded. New plasma samples were collected in Ljubljana (Slovenia) and Vienna (Austria) for the discovery phase and only in Ljubljana for the replication. Samples were measured with Biocrates p180 kit (37) in 287 plasma samples (discovery study) and in 245 plasma samples (replication study) obtained from controls and endometriosis patients at different stages. All measurements and primary data underwent quality assurance procedures and only a validated data set was used for biostatistical analyses.

The data was calculated with R 4.0.2 (2020 Jun. 22). Biostatistics analyses revealed no significant differences in age, BMI or menstrual cycle between control and case groups.

### Quality Control Elements

NA imputation (missing data imputation) was performed for metabolites with less than 40% missing values. Metabolites with more than 40% missing values were discarded.

Further, remaining metabolites were checked for coefficients of variation (CV %) of more than 25% and the affected metabolites discarded from the data set. The data was also log-transformed in order to check for lognormal data distribution by Shapiro-Wilks test, but log-normal data distribution was not detected.

### PLS-DA Analysis Case Vs Control

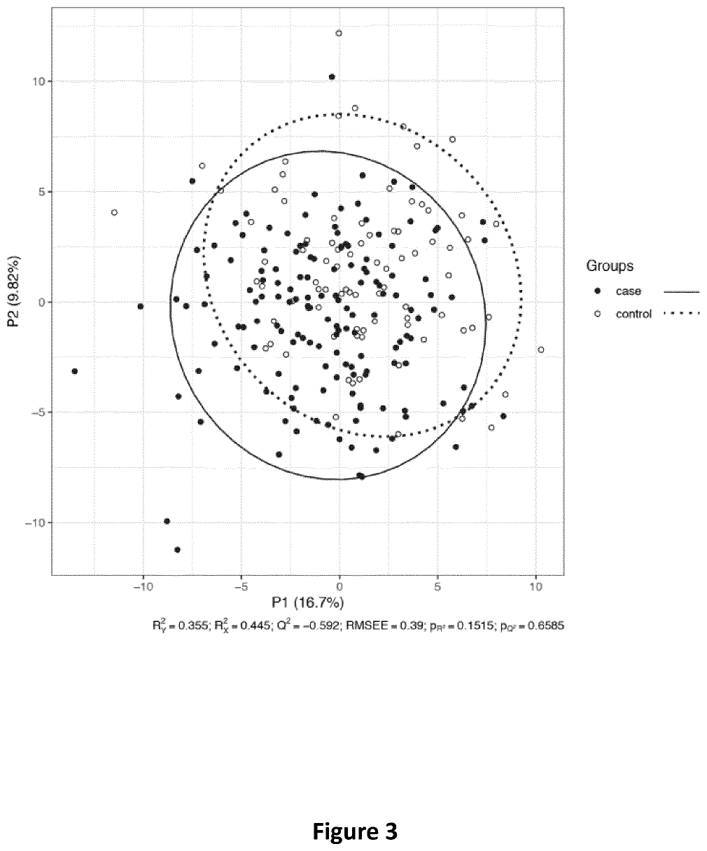

Non-log transformed metabolite data was used to calculate the PLS-DA as given above in FIG. 3.

This PLS-DA does not include the metabolites which were found to be above the CV % threshold of 25% or were excluded due to being above the NA threshold of 40% (as described before). As is evident from the PLS-DA statistics, a separation by group is not possible if based on absolute concentrations of metabolites.

### Dedicated Analysis for Impact of Confounders on Plasma Metabolome

Analyses from log transforming and autoscaling the metabolite concentrations of all samples for confounder effect like menstrual cycle, age, BMI, presence of other disease (cancer or diabetes) or medication revealed no detectable impact.

Statistics Control Vs Case Groups with False Discovery Rate

Log-transformation of the data does, according to Shapiro-Wilks test, lead to non-parametric data. Therefore, Mann-Whitney-U tests were performed on the data. When performing multiple testing correction by the FDR method, these significant results can be found. Without multiple testing correction, more metabolites appear to be significantly different.

Calculation of ROC and AUC with GLM Models

As the classic statistical approach using absolute metabolite concentrations proved not sufficient for the data set presented, the metabolite selection was performed by machine learning with randomForest (RF) on all metabolites and all possible metabolite ratios.

All calculations are performed on the 10× cross validated data—this means data was randomly divided into 66% training data and 34% test data for each cross validation step.

In order to narrow down the possible candidates for further modelling with generalized linear models (GLM) and to obtain reporter-operator curves (ROC) with area under the curve (AUC) calculations, the following criteria were used for RF:
Number of calculated trees: 500
MeanDecreaseGini: >0

From the remaining candidates only those in the top 10% of the performance were selected.

From the remaining candidates all possible combinations for 3-predictor model for the GLM were calculated. This results in 67599 possible combinations for these GLMs when leaving out metabolites/metabolite ratios which are derived from total sums of measured metabolites assigned within specific chemical classes. The later would be impractical to measure in a diagnostic assay and were excluded.

The GLMs were calculated on the response of samples being in the control group or case group. Modelling with disease stage or disease type as response lead to over-fitting of the models.

Although the ROCs with their respective AUCs shown in the following pages only show an AUC up to average 0.82 in the test data set, it is still worth to note that it is very well possible to distinguish the responses in the models with a rather fair accuracy by selecting the parameters of the glms by RF from all the possible metabolites and ratios. This is not a feasible approach for PLS-DA analysis due to the high likelihood of over-fitting the model.

### Description of GLM Models and Metabolites

The following chapters describe the GLMs identified for specific forms of endometriosis. The GLMs are annotated for performance (AUC and RMSE). The metabolites constituting the GLMs are extracted and annotated. Further the basis for diagnostic decisions is provided.

### 1. All Types of Endometriosis

TABLE 3
GLMs for all types of endometriosis

| | | AUC | |
|---|---|---|---|
| # | GLM | average | RMSE |
| 1 | LysoPC a C17:0_div_by_SM(OH) C16:1 + Arg_div_by_PC ae C36:0 + PC | 0.7256 | 0.0427 |
| | ae C38:0_div_by_PC ae C40:0 | | |
| 2 | Arg_div_by_PC ae C36:0 + lysoPC a C16:0_div_by_SM C18:1 + PC ae | 0.7240 | 0.0434 |
| | C38:0_div_by_PC ae C40:0 | | |
| 3 | Thr_div_by_PC aa C34:3 + LysoPC a C17:0_div_by_SM(OH) C16:1 + | 0.7159 | 0.0767 |
| | Arg_div_by_PC ae C36:0 | | |
| 4 | Thr_div_by_PC aa C34:3 + Arg_div_by_PC ae C36:0 + lysoPC a | 0.7120 | 0.0713 |
| | C16:0_div_by_SM C18:1 | | |
| 5 | Arg_div_by_PC aa C36:6 + LysoPC a C17:0_div_by_SM(OH) C16:1 + | 0.7087 | 0.0544 |
| | Arg_div_by_PC ae C36:0 | | |
| 6 | LysoPC a C17:0_div_by_SM(OH) C16:1 + Arg_div_by_PC ae C36:0 + | 0.7061 | 0.0540 |
| | C18_div_by_lysoPC a C14:0 | | |
| 7 | lysoPC a C16:0_div_by_SM C18:1 + PC ae C38:0_div_by_PC ae C40:0 + | 0.7040 | 0.0402 |
| | Ser_div_by_PC ae C44:3 | | |
| 8 | LysoPC a C17:0_div_by_SM(OH) C16:1 + Arg_div_by_PC ae C36:0 + | 0.7037 | 0.0786 |
| | Trp_div_by_PC ae C38:3 | | |
| 9 | Arg_div_by_PC ae C36:0 + lysoPC a C16:0_div_by_SM C18:1 + | 0.7029 | 0.0559 |
| | C8_div_by_PC ae C30:0 | | |
| 10 | Thr_div_by_PC ae C36:5 + Arg_div_by_PC ae C36:0 + lysoPC a | 0.7021 | 0.0503 |
| | C16:0_div_by_SM C18:1 | | |
| 11 | Arg_div_by_PC ae C36:0 + C18_div_by_lysoPC a C14:0 + lysoPC a | 0.7013 | 0.0531 |
| | C16:0_div_by_SM C18:1 | | |
| 12 | Arg_div_by_PC ae C36:0 + C10_div_by_PC ae C38:6 + lysoPC a | 0.7006 | 0.0465 |
| | C16:0_div_by_SM C18:1 | | |
| 13 | Arg_div_by_PC aa C36:6 + Arg_div_by_PC ae C36:0 + lysoPC a | 0.7002 | 0.0523 |
| | C16:0_div_by_SM C18:1 | | |
| 14 | Tyr_div_by_PC aa C42:4 + C3-DC_div_by_C18 + PC aa C42:1_div_by_SM | 0.7397 | 0.0906 |
| | C22:3 | | |
| 15 | C3-DC_div_by_C18 + PC aa C42:1_div_by_SM C22:3 + C6 (C4:1- | 0.7290 | 0.0969 |
| | DC)_div_by_SM C16:1 | | |

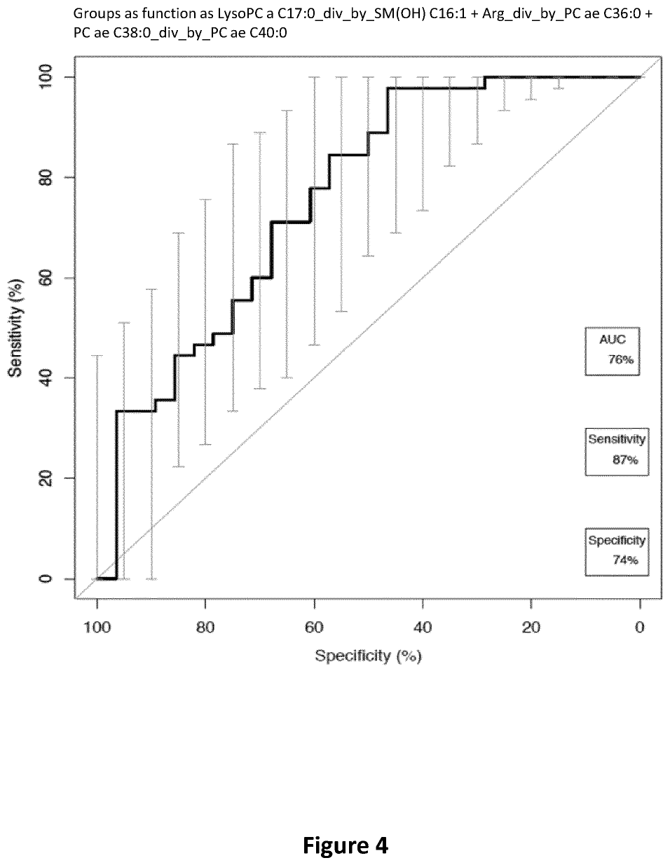

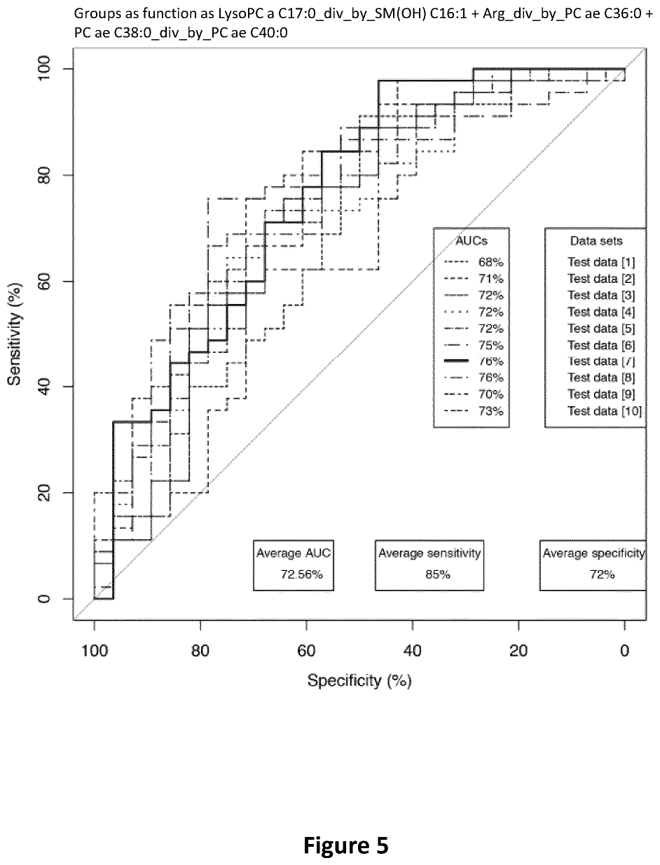

GLM describes a model formula consisting of sum of three metabolite ratios. The models are listed according to average AUC (Area Under the curve) average and the RMSE (Root Mean Squared Error) less than 0.15. The AUC analyses for best model and its cross-validation are presented in FIGS. 4 and 5.

TABLE 4
Performance of GLM models for all types of endometriosis

| GLM | AUC | Sensitivity | Specificity | AUC | Sensitivity | Specificity |
|---|---|---|---|---|---|---|
| model | best | best | best | average | average | average |
| 1 | 0.76 | 0.87 | 0.74 | 0.72 | 0.85 | 0.72 |
| 2 | 0.76 | 0.84 | 0.72 | 0.72 | 0.87 | 0.73 |
| 3 | 0.83 | 0.84 | 0.78 | 0.71 | 0.88 | 0.71 |
| 4 | 0.82 | 0.84 | 0.76 | 0.71 | 0.87 | 0.71 |
| 5 | 0.76 | 0.87 | 0.74 | 0.70 | 0.86 | 0.71 |
| 6 | 0.75 | 0.87 | 0.74 | 0.70 | 0.88 | 0.72 |
| 7 | 0.75 | 0.36 | 0.67 | 0.70 | 0.73 | 0.67 |
| 8 | 0.75 | 0.84 | 0.75 | 0.70 | 0.87 | 0.71 |
| 9 | 0.73 | 0.82 | 0.76 | 0.70 | 0.82 | 0.68 |
| 10 | 0.76 | 0.87 | 0.74 | 0.70 | 0.87 | 0.71 |
| 11 | 0.75 | 0.84 | 0.70 | 0.70 | 0.85 | 0.70 |
| 12 | 0.73 | 0.89 | 0.74 | 0.70 | 0.86 | 0.70 |
| 13 | 0.72 | 0.89 | 0.73 | 0.70 | 0.87 | 0.71 |
| 14 | 0.84 | 0.77 | 0.68 | 0.74 | 0.68 | 0.65 |
| 15 | 0.83 | 0.84 | 0.64 | 0.72 | 0.66 | 0.65 |

Only for the first ten best GLM models the DxS values are calculated. In the development of GLMs we observed that further models, analysed for all types of endometriosis, are not contributing to the phenotype explanation significantly. In fact, we noticed that the performance drops continuously after several iterations, especially after the 10^{th} model.

TABLE 5
Interpretation basis for diagnosis of all types endometriosis

| | Reference value | | DxS - Fold |
|---|---|---|---|
| GLM model | (Value for Control) | Value for Case | change Log2 |
| 1 | 114.07 | 99.48 | 0.20 |
| 2 | 119.59 | 105.58 | 0.18 |
| 3 | 123.88 | 109.73 | 0.17 |
| 4 | 129.40 | 115.84 | 0.16 |
| 5 | 258.34 | 237.52 | 0.12 |
| 6 | 113.75 | 99.18 | 0.20 |
| 7 | 978.17 | 884.20 | 0.15 |
| 8 | 135.71 | 121.58 | 0.16 |
| 9 | 119.63 | 105.65 | 0.18 |
| 10 | 131.28 | 117.05 | 0.17 |

A numeric value is calculated according to the GLM model formula. The calculated value is used to discriminate between diseased and not affected patient. Negative or positive values of DxS describe the direction of differences of case versus control.

### 2. Peritoneal Endometriosis

TABLE 6
GLMs for peritoneal endometriosis

| | | AUC | |
|---|---|---|---|
| # | GLM | average | RMSE |
| 1 | lysoPC a C16:0_div_by_SM(OH) C16:1 + PC aa C32:0_div_by_SM C18:0 + | 0.8080 | 0.08836 |
| | PC aa C32:0_div_by_PC aa C38:3 | | |
| 2 | lysoPC a C16:0_div_by_SM(OH) C16:1 + PC aa C32:0_div_by_SM C18:0 + | 0.7949 | |
| | Arg_div_by_PC ae C34:0 | | 0.09458 |
| 3 | C5-M-DC_div_by_PC aa C42:5 + Arg_div_by_PC ae C34:0 + lysoPC a | 0.7901 | |
| | C18:2_div_by_PC ae C40:6 | | 0.10012 |
| 4 | lysoPC a C18:2_div_by_PC ae C40:4 + PC ae C40:6_div_by_CPT I ratio + | 0.7875 | |
| | lysoPC a C17:0_div_by_SM C18:0 | | 0.08568 |
| 5 | C4_div_by_PC ae C30:2 + Arg_div_by_PC ae C34:0 + lysoPC a | 0.7842 | 0.10440 |
| | C18:2_div_by_PC ae C40:6 | | |
| 6 | PC ae C40:6_div_by_CPT \| ratio + C4_div_by_PC ae C30:2 + lysoPC a | 0.7839 | 0.07258 |
| | C18:2_div_by_PC ae C40:6 | | |
| 7 | lysoPC a C18:2_div_by_PC ae C40:4 + Arg_div_by_PC ae C34:0 + PC ae | 0.7806 | 0.08948 |
| | C34:1_div_by_PC ae C42:0 | | |

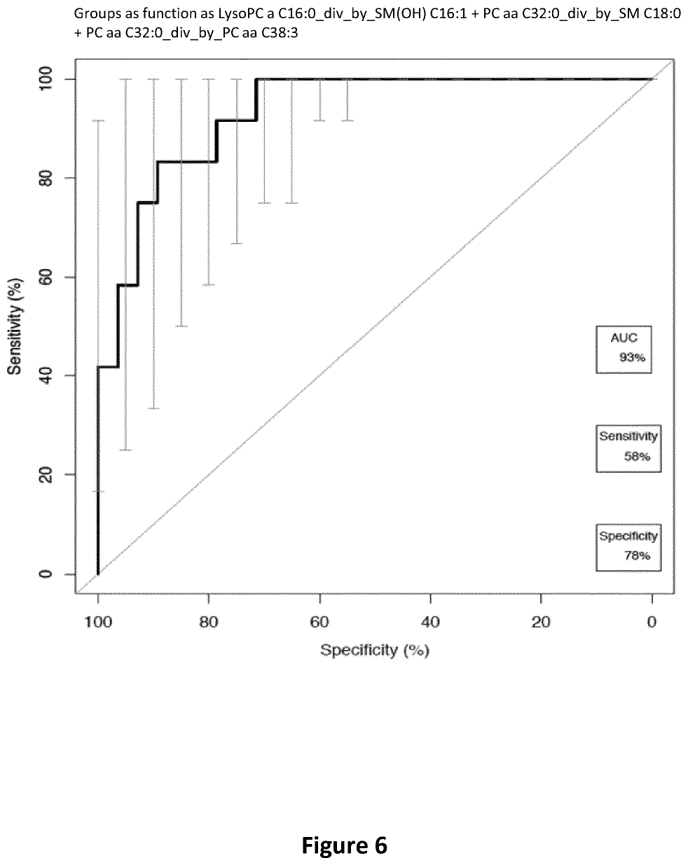

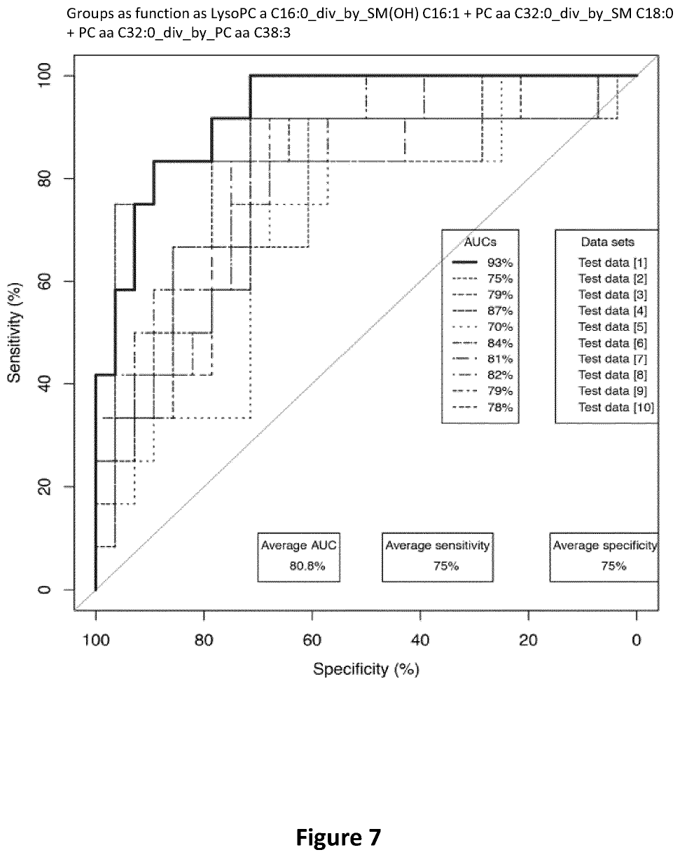

GLM describes a model formula consisting of sum of three metabolite ratios. The models are listed according to average AUC (Area Under the curve) average and the RMSE (Root Mean Squared Error) less than 0.15. The AUC analyses for best model and its cross-validation are presented in FIGS. 6 and 7.

TABLE 7
Performance of GLM models for peritoneal endometriosis

| GLM | AUC | Sensitivity | Specificity | AUC | Sensitivity | Specificity |
|---|---|---|---|---|---|---|
| model | best | best | best | average | average | average |
| 1 | 0.93 | 0.58 | 0.78 | 0.80 | 0.75 | 0.75 |
| 2 | 0.90 | 0.42 | 0.71 | 0.79 | 0.74 | 0.75 |
| 3 | 0.93 | 0.89 | 0.83 | 0.79 | 0.79 | 0.78 |
| 4 | 0.91 | 0.83 | 0.83 | 0.78 | 0.71 | 0.77 |
| 5 | 0.91 | 0.67 | 0.73 | 0.78 | 0.77 | 0.74 |
| 6 | 0.88 | 0.75 | 0.75 | 0.78 | 0.79 | 0.76 |
| 7 | 0.89 | 0.67 | 0.73 | 0.78 | 0.71 | 0.71 |

Only for the seven best GLM models the DxS values are calculated. In the development of GLMs we observed that further models, analysed for peritoneal endometriosis, are not contributing to the phenotype explanation significantly. In fact, we noticed that the performance drops continuously after several iterations, especially after the 7^{th} model.

TABLE 8
Interpretation basis for diagnosis of peritoneal endometriosis

| | Reference value | | DxS - Fold |
|---|---|---|---|
| GLM model | (Value for Control) | Value for Case | change Log2 |
| 1 | 20.32 | 27.15 | −0.42 |
| 2 | 20.20 | 27.08 | −0.42 |
| 3 | 95.57 | 101.91 | −0.09 |
| 4 | 19.90 | 26.69 | −0.42 |
| 5 | 95.64 | 102.05 | −0.09 |
| 6 | 19.61 | 26.42 | −0.43 |
| 7 | 85.00 | 87.59 | −0.04 |

A numeric value is calculated according to the GLM model formula. The calculated value is used to discriminate between diseased and not affected patient. Negative or positive values of DxS describe the direction of differences of case versus control.

### 3. Peritoneal Mixed Endometriosis

TABLE 9
GLMs for peritoneal mixed endometriosis

| | | AUC | |
|---|---|---|---|
| # | GLM | average | RMSE |
| 1 | Orn_div_by_PC ae C38:0 + C4_div_by_PC aa C38:4 + Tyr_div_by_PC aa | 0.6805 | 0.0851 |
| | C42:2 | | |
| 2 | Arg_div_by_PC aa C36:6 + C5_div_by_lysoPC a C17:0 + C5_div_by_Arg | 0.6793 | 0.0860 |
| 3 | Orn_div_by_PC ae C38:0 + C5_div_by_lysoPC a C17:0 + C5_div_by_Arg | 0.6721 | 0.0822 |
| 4 | CO_div_by_Gly + Orn_div_by_PC ae C38:0 + Tyr_div_by_PC aa C42:2 | 0.6683 | 0.0648 |
| 5 | SM C18:0 + C5_div_by_lysoPC a C17:0 + C5_div_by_Arg | 0.6676 | 0.0956 |
| 6 | C5_div_by_lysoPC a C17:0 + C5_div_by_Arg + Ser_div_by_SM(OH) C16:1 | 0.6669 | 0.0843 |
| 7 | SM C18:0 + CO_div_by_Gly + Tyr_div_by_PC aa C42:2 | 0.6668 | 0.0961 |
| 8 | Orn_div_by_PC ae C38:0 + C3_div_by_PC ae C40:5 + Tyr_div_by_PC aa | 0.6662 | 0.0764 |
| | C42:2 | | |
| 9 | Pro_div_by_PC ae C34:0 + Orn_div_by_PC ae C38:0 + Tyr_div_by_PC aa | 0.6649 | 0.0725 |
| | C42:2 | | |
| 10 | C4_div_by_Ser + Orn_div_by_PC ae C38:0 + Tyr_div_by_PC aa C42:2 | 0.6645 | 0.1073 |
| 11 | SM C18:0 + Orn_div_by_PC ae C38:0 + Tyr_div_by_PC aa C42:2 | 0.6639 | 0.0760 |
| 12 | Orn_div_by_PC ae C38:0 + C4_div_by_PC ae C40:3 + Tyr_div_by_PC aa | 0.6637 | 0.1018 |
| | C42:2 | | |
| 13 | Orn_div_by_PC ae C38:0 + PC ae C42:3_div_by_SM(OH) C16:1 + | 0.6611 | 0.0700 |
| | Tyr_div_by_PC aa C42:2 | | |
| 14 | Orn_div_by_PC ae C38:0 + Tyr_div_by_PC ae C38:0 + Tyr_div_by_PC aa | 0.6581 | 0.0702 |
| | C42:2 | | |
| 15 | Gly_div_by_SM C24:1 + PC aa C32:0_div_by_PC aa C40:1 + PC aa | | |
| | C36:4_div_by_PC aa C38:0 | | |
| 16 | Gly_div_by_PC aa C42:5 + PC aa C36:4_div_by_PC aa C38:0 + CO_div_by_{—} | | |
| | SM(OH) C22:2 | | |

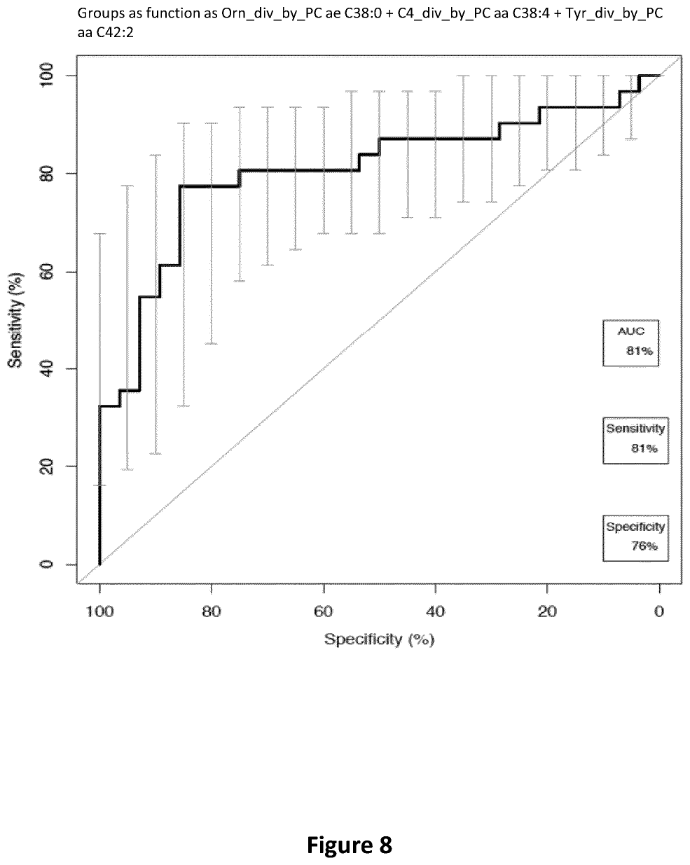

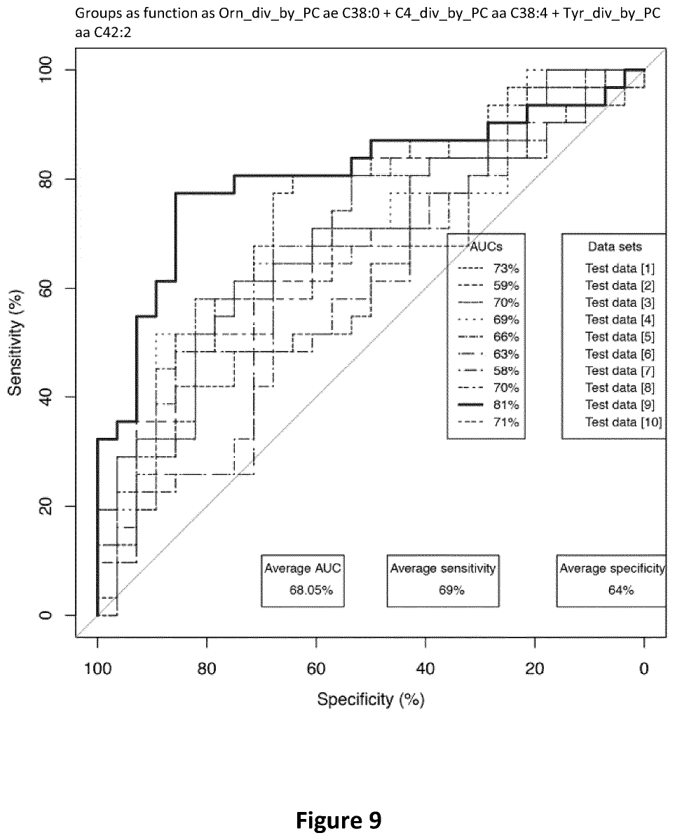

GLM describes a model formula consisting of sum of three metabolite ratios. The models are listed according to average AUC (Area Under the curve) average and the RMSE (Root Mean Squared Error) less than 0.15. The AUC analyses for best model and its cross-validation are presented in FIGS. 8 and 9.

TABLE 10
Performance of GLM models peritoneal mixed endometriosis

| GLM | AUC | Sensitivity | Specificity | AUC | Sensitivity | Specificity |
|---|---|---|---|---|---|---|
| model | best | best | best | average | average | average |
| 1 | 0.81 | 0.81 | 0.76 | 0.68 | 0.69 | 0.64 |
| 2 | 0.77 | 0.74 | 0.70 | 0.67 | 0.73 | 0.73 |
| 3 | 0.75 | 0.74 | 0.74 | 0.67 | 0.71 | 0.65 |
| 4 | 0.74 | 0.68 | 0.63 | 0.66 | 0.67 | 0.64 |
| 5 | 0.79 | 0.77 | 0.80 | 0.66 | 0.69 | 0.63 |
| 6 | 0.78 | 0.71 | 0.76 | 0.66 | 0.68 | 0.63 |
| 7 | 0.80 | 0.77 | 0.75 | 0.66 | 0.70 | 0.64 |
| 8 | 0.80 | 0.87 | 0.73 | 0.66 | 0.68 | 0.62 |
| 9 | 0.78 | 0.81 | 0.68 | 0.66 | 0.70 | 0.63 |
| 10 | 0.79 | 0.77 | 0.75 | 0.66 | 0.70 | 0.65 |

Only for the first ten best GLM models the DxS values are calculated. In the development of GLMs we observed that further models, analysed for peritoneal mixed endometriosis, are not contributing to the phenotype explanation significantly. In fact, we noticed that the performance drops continuously after several iterations, especially after the 10^{th} model.

TABLE 11
Interpretation basis for diagnosis of peritoneal mixed endometriosis

| | Reference value | | DxS - Fold |
|---|---|---|---|
| GLM model | (Value for Control) | Value for Case | change Log2 |
| 1 | 293.09 | 265.04 | 0.15 |
| 2 | 139.35 | 134.95 | 0.05 |
| 3 | 32.90 | 34.57 | −0.07 |
| 4 | 293.22 | 265.17 | 0.15 |
| 5 | 23.65 | 22.41 | 0.08 |
| 6 | 38.99 | 38.51 | 0.02 |
| 7 | 283.96 | 253.01 | 0.17 |
| 8 | 293.18 | 265.13 | 0.15 |
| 9 | 424.62 | 397.46 | 0.10 |
| 10 | 293.09 | 265.04 | 0.15 |

A numeric value is calculated according to the GLM model formula. The calculated value is used to discriminate between diseased and not affected patient. Negative or positive values of DxS describe the direction of differences of case versus control.

### 4. Ovarian Endometriosis

TABLE 12
GLMs for ovarian endometriosis

| | | AUC | |
|---|---|---|---|
| # | GLM | average | RMSE |
| 1 | PC aa C36:3_div_by_PC ae C40:5 + lysoPC a C14:0_div_by_PC aa C28:1 + | 0.7111 | 0.0776 |
| | Met_div_by_PC aa C36:3 | | |
| 2 | PC aa C38:0_div_by_PC ae C36:1 + Thr_div_by_ SM (OH) C22:1+ lysoPC | 0.6992 | 0.0782 |
| | a C14:0_div_by_PC aa C28:1 | | |
| 3 | Thr_div_by_ SM (OH) C22:1 + PC aa C28:1_div_by_PC ae C34:3 + | 0.6992 | 0.0982 |
| | C18:2_div_by_PC ae C34:3 | | |
| 4 | PC aa C36:3_div_by_PC ae C40:5 + C3_div_by_PC ae C34:1 + | 0.6952 | 0.0893 |
| | Met_div_by_PC aa C36:3 | | |
| 5 | PC aa C36:3_div_by_PC ae C40:5 + PC aa C28:1_div_by_PC ae C34:3 + | 0.6952 | 0.0414 |
| | Gly_div_by_PC ae C36:1 | | |
| 6 | PC aa C38:0_div_by_PC ae C36:1 + C18:2_div_by_PC ae C34:3 + | 0.6948 | 0.1026 |
| | Met_div_by_PC aa C36:3 | | |
| 7 | PC aa C38:0_div_by_PC ae C36:1 + C10:1_div_by_PC aa C36:1 + PC ae | 0.6928 | 0.0838 |
| | C38:3_div_by_SM C18:1 | | |
| 8 | PC aa C38:0_div_by_PC ae C36:1 + Gly_div_by_PC ae C36:1 + | 0.6928 | 0.0712 |
| | C3_div_by_PC ae C34:1 | | |
| 9 | PC aa C38:0_div_by_PC ae C36:1 + C12-DC_div_by_C14:2 + PC ae | 0.6924 | 0.1018 |
| | C38:3_div_by_SM C18:1 | | |
| 10 | PC aa C36:3_div_by_PC ae C40:5 + PC aa C38:3_div_by_PC ae C44:5 + | 0.6920 | 0.1008 |
| | Met_div_by_PC aa C36:3 | | |
| 11 | PC aa C38:0_div_by_PC ae C36:1 + PC ae C38:3_div_by_SM C18:1 + | 0.6916 | 0.0997 |
| | Met_div_by_PC aa C36:3 | | |
| 12 | PC aa C28:1_div_by_PC ae C34:3 + C18:2_div_by_PC ae C34:3 + | 0.6912 | 0.1323 |
| | C4_div_by_C5:1 | | |
| 13 | PC aa C36:3_div_by_PC ae C40:5 + lysoPC a C20:4_div_by_PC ae C32:1 + | 0.6912 | 0.0941 |
| | Met_div_by_PC aa C36:3 | | |
| 14 | PC aa C36:3_div_by_PC ae C40:5 + lysoPC a C20:4_div_by_PC aa C32:3 + | 0.6900 | 0.1029 |
| | Met_div_by_PC aa C36:3 | | |

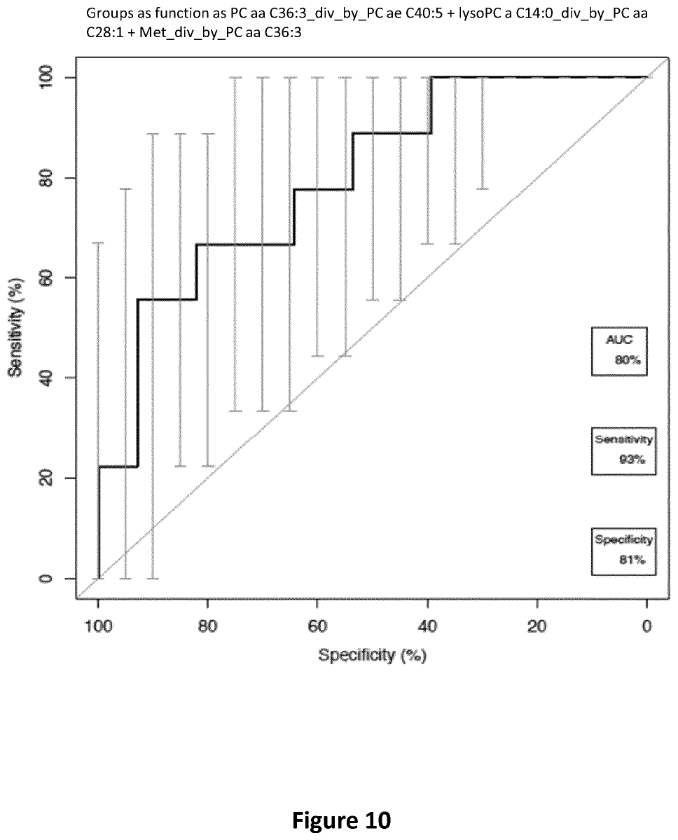

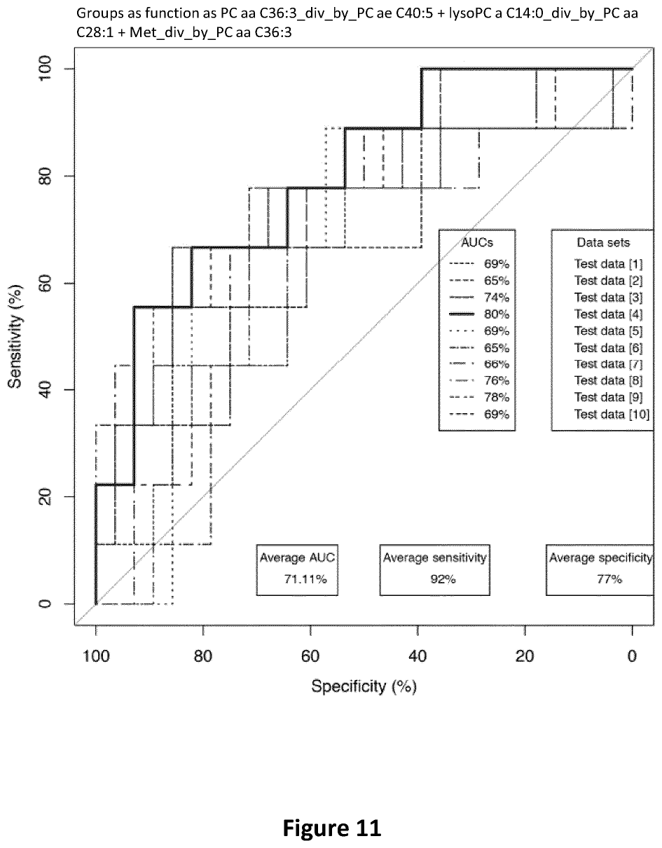

GLM describes a model formula consisting of sum of three metabolite ratios. The models are listed according to average AUC (Area Under the curve) average and the RMSE (Root Mean Squared Error) less than 0.15. The AUC analyses for best model and its cross-validation are presented in FIGS. 10 and 11.

TABLE 13
Performance of GLM models for ovarian endometriosis

| GLM | AUC | Sensitivity | Specificity | AUC | Sensitivity | Specificity |
|---|---|---|---|---|---|---|
| model | best | best | best | average | average | average |
| 1 | 0.80 | 0.93 | 0.81 | 0.71 | 0.92 | 0.77 |
| 2 | 0.82 | 0.96 | 0.82 | 0.69 | 0.77 | 0.73 |
| 3 | 0.77 | 0.96 | 0.82 | 0.69 | 0.92 | 0.80 |
| 4 | 0.80 | 0.89 | 0.81 | 0.69 | 0.97 | 0.78 |
| 5 | 0.72 | 0.93 | 0.76 | 0.69 | 0.97 | 0.77 |
| 6 | 0.86 | 0.93 | 0.84 | 0.69 | 0.73 | 0.80 |
| 7 | 0.80 | 0.89 | 0.78 | 0.69 | 0.97 | 0.79 |
| 8 | 0.82 | 0.22 | 0.50 | 0.69 | 0.90 | 0.75 |
| 9 | 0.80 | 0.93 | 0.79 | 0.69 | 0.82 | 0.73 |
| 10 | 0.81 | 0.93 | 0.79 | 0.69 | 0.98 | 0.77 |

Only for the first ten best GLM models the DxS values are calculated. In the development of GLMs we observed that further models, analysed for ovarian endometriosis, are not contributing to the phenotype explanation significantly. In fact, we noticed that the performance drops continuously after several iterations, especially after the 10^{th} model.

TABLE 14
Interpretation basis for diagnosis of ovarian endometriosis

| | Reference value | | DxS - Fold |
|---|---|---|---|
| GLM model | (Value for Control) | Value for Case | change Log2 |
| 1 | 37.18 | 35.89 | 0.05 |
| 2 | 9.66 | 8.98 | 0.10 |
| 3 | 8.42 | 7.61 | 0.15 |
| 4 | 36.07 | 34.72 | 0.06 |
| 5 | 88.29 | 82.40 | 0.10 |
| 6 | 0.77 | 0.80 | −0.05 |
| 7 | 0.84 | 0.88 | −0.08 |
| 8 | 52.63 | 48.16 | 0.13 |
| 9 | 4.35 | 3.78 | 0.20 |
| 10 | 56.62 | 55.51 | 0.03 |

A numeric value is calculated according to the GLM model formula. The calculated value is used to discriminate between diseased and not affected patient. Negative or positive values of DxS describe the direction of differences of case versus control.

### Ovarian Mixed Endometriosis

TABLE 15
GLMs for ovarian mixed endometriosis

| | | AUC | |
|---|---|---|---|
| # | GLM | average | RMSE |
| 1 | C10_div_by_PC aa C36:6 + Pro_div_by_PC ae C34:0 + PC ae | 0.6656 | 0.0717 |
| | C42:3_div_by_SM(OH) C16:1 | | |
| 2 | C10_div_by_PC aa C36:6 + PC ae C42:3_div_by_SM(OH) C16:1+ | 0.6612 | 0.0689 |
| | C6:1_div_by_lysoPC a C20:4 | | |
| 3 | C10_div_by_PC aa C36:6 + PC ae C42:3_div_by_SM(OH) C16:1+ lysoPC a | 0.6593 | 0.0710 |
| | C20:4_div_by_PC ae C40:2 | | |
| 4 | Ser_div_by_PC aa C38:3 + C10_div_by_PC aa C36:6 + PC ae | 0.6590 | 0.0789 |
| | C42:3_div_by_SM(OH) C16:1 | | |
| 5 | C10_div_by_lysoPC a C18:1 + C10_div_by_PC aa C36:6 + PC ae | 0.6562 | 0.1236 |
| | C42:3_div_by_SM(OH) C16:1 | | |
| 6 | C10_div_by_PC aa C36:6 + lysoPC a C24:0_div_by_PC ae C42:3 + PC ae | 0.6548 | 0.1142 |
| | C42:3_div_by_SM(OH) C16:1 | | |
| 7 | C10_div_by_PC aa C36:6 + lysoPC a C18:1_div_by_PC aa C36:1 + PC ae | 0.6537 | 0.0830 |
| | C42:3_div_by_SM(OH) C16:1 | | |
| 8 | C10_div_by_PC aa C36:6 + Gly_div_by_PC ae C34:1 + PC ae | 0.6517 | 0.0746 |
| | C42:3_div_by_SM(OH) C16:1 | | |
| 9 | Gln_div_by_PC ae C30:2 + C10_div_by_PC aa C36:6 + PC ae | 0.6504 | 0.0846 |
| | C42:3_div_by_SM(OH) C16:1 | | |
| 10 | Pro_div_by_PC ae C34:0 + lysoPC a C24:0_div_by_PC ae C42:3 + PC ae | 0.6474 | 0.0399 |
| | C42:3_div_by_SM(OH) C16:1 | | |
| 11 | C10:1_div_by_lysoPC a C24:0 + lysoPC a C24:0_div_by_PC ae C42:3 + PC | 0.6386 | 0.0736 |
| | ae C42:3_div_by_ SM(OH) C16:1 | | |
| 12 | PC ae C44:3_div_by_CPT.I.ratio + PC ae C34:0_div_by_PC ae C40:3 + | 0.7308 | 0.0868 |
| | C16:2-OH_div_by_SM C20:2 | | |
| 13 | PC ae C44:6_div_by_SM C22:3 + PC ae C34:0_div_by_PC ae C40:3 + | 0.7045 | 0.1262 |
| | C10:1_div_by_C14:2-OH | | |

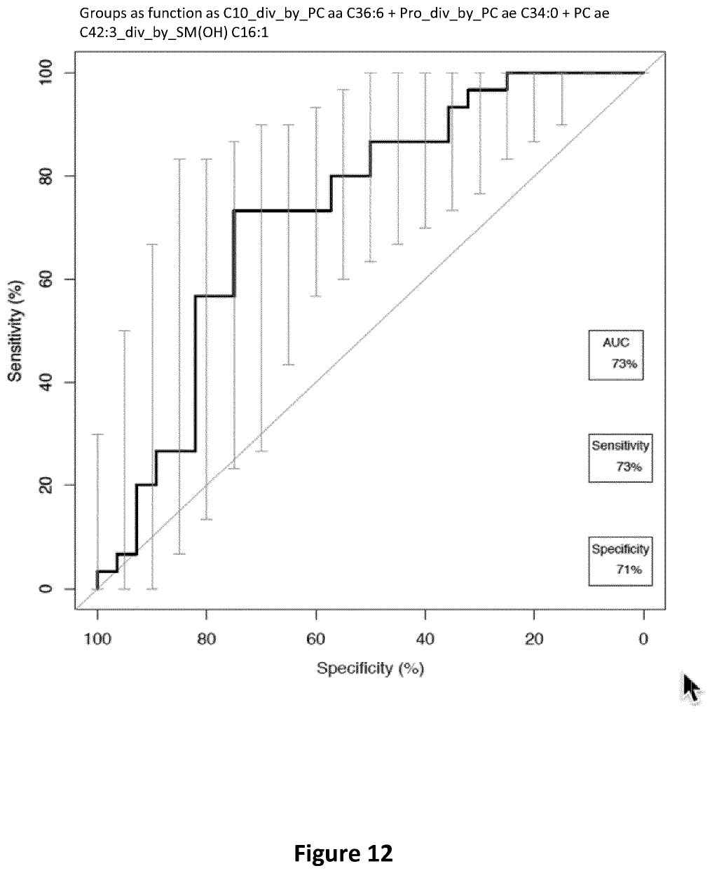

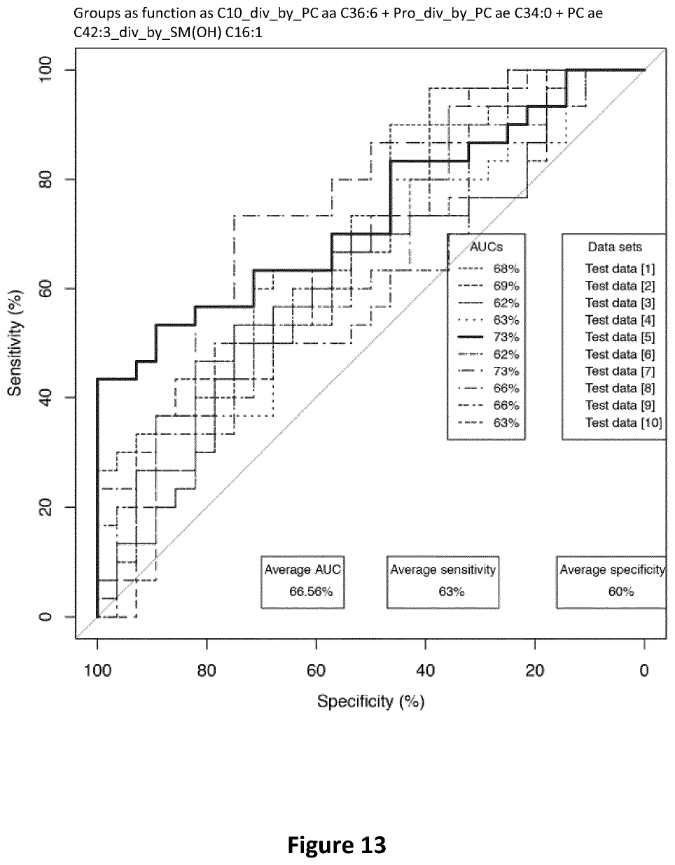

GLM describes a model formula consisting of sum of three metabolite ratios. The models are listed according to average AUC (Area Under the curve) average and the RMSE (Root Mean Squared Error) less than 0.15. The AUC analyses for best model and its cross-validation are presented in FIGS. 12 and 13.

TABLE 16
Performance of GLM models ovarian mixed endometriosis

| GLM | AUC | Sensitivity | Specificity | AUC | Sensitivity | Specificity |
|---|---|---|---|---|---|---|
| model | best | best | best | average | average | average |
| 1 | 0.73 | 0.73 | 0.71 | 0.66 | 0.63 | 0.60 |
| 2 | 0.72 | 0.64 | 0.62 | 0.66 | 0.61 | 0.61 |
| 3 | 0.71 | 0.68 | 0.58 | 0.65 | 0.61 | 0.60 |
| 4 | 0.76 | 0.77 | 0.72 | 0.65 | 0.58 | 0.58 |
| 5 | 0.77 | 0.73 | 0.76 | 0.65 | 0.63 | 0.63 |
| 6 | 0.76 | 0.68 | 0.61 | 0.65 | 0.64 | 0.60 |
| 7 | 0.72 | 0.64 | 0.62 | 0.65 | 0.61 | 0.59 |
| 8 | 0.71 | 0.67 | 0.67 | 0.65 | 0.61 | 0.59 |

Only for the first eight best GLM models the DxS values are calculated. In the development of GLMs we observed that further models, for analysed mixed ovarian endometriosis, are not contributing to the phenotype explanation significantly. In fact, we noticed that the performance drops continuously after several iterations, especially after the 8^{th} model.

TABLE 17
Interpretation basis for diagnosis of ovarian mixed endometriosis

| | Reference value | | DxS - Fold |
|---|---|---|---|
| GLM model | (Value for Control) | Value for Case | change Log2 |
| 1 | 135.99 | 129.81 | 0.07 |
| 2 | 0.71 | 0.83 | −0.23 |
| 3 | 3.94 | 4.04 | −0.04 |
| 4 | 4.13 | 4.17 | −0.01 |
| 5 | 0.73 | 0.85 | −0.22 |
| 6 | 1.47 | 1.61 | −0.13 |
| 7 | 1.13 | 1.26 | −0.15 |
| 8 | 37.37 | 37.21 | 0.01 |

A numeric value is calculated according to the GLM model formula. The calculated value is used to discriminate between diseased and not affected patient. Negative or positive values of DxS describe the direction of differences of case versus control.

### LIST OF REFERENCES CITED IN THE DESCRIPTION

1. Giudice L C. Clinical practice. Endometriosis. N Engl J Med. 2010; 362(25):2389-98.
2. Giudice L C, Kao L C. Endometriosis. Lancet. 2004; 364(9447):1789-99.
3. Sourial S, Tempest N, Hapangama D K. Theories on the pathogenesis of endometriosis. Int J Reprod Med. 2014; 2014:179515.
4. Burney R O, Giudice L C. Pathogenesis and pathophysiology of endometriosis. Fertil Steril. 2012; 98(3):511-9.
5. Goenka L, George M, Sen M. A peek into the drug development scenario of endometriosis—A systematic review. Biomed Pharmacother. 2017; 90:575-85.
6. Acien P, Velasco I. Endometriosis: a disease that remains enigmatic. ISRN Obstet Gynecol. 2013; 2013:242149.
7. Nnoaham K E, Hummelshoj L, Webster P, d'Hooghe T, de Cicco Nardone F, de Cicco Nardone C, et al. Impact of endometriosis on quality of life and work productivity: a multicenter study across ten countries. Fertil Steril. 2011; 96(2):366-73 e8.
8. May K E, Conduit-Hulbert S A, Villar J, Kirtley S, Kennedy S H, Becker C M. Peripheral biomarkers of endometriosis: a systematic review. Hum Reprod Update. 2010; 16(6):651-74.
9. Rizner T L. Noninvasive biomarkers of endometriosis: myth or reality?Expert Rev Mol Diagn. 2014; 14(3):365-85.
10. Farquhar C. Endometriosis. BMJ. 2007; 334(7587):249-53.
11. Nisenblat V, Prentice L, Bossuyt P M, Farquhar C, Hull M L, Johnson N. Combination of the non-invasive tests for the diagnosis of endometriosis. Cochrane Database Syst Rev. 2016; 7:CD012281.
12. Walter A J, Hentz J G, Magtibay P M, Cornella J L, Magrina J F. Endometriosis: correlation between histologic and visual findings at laparoscopy. Am J Obstet Gynecol. 2001; 184(7):1407-11; discussion 11-3.
13. Almeida Filho D P, Oliveira U, Amaral V F. Accuracy of laparoscopy for assessing patients with endometriosis. Sao Paulo Med J. 2008; 126(6):305-8.
14. Wykes C B, Clark J T, Khan K S. Accuracy of laparoscopy in the diagnosis of endometriosis: a systematic quantitative review. International Journal of Obstetrics and Gynaecology. 2004; 111:1204-12.
15. Zondervan K T, Becker C M, Koga K, Missmer S A, Taylor R N, Vigano P. Endometriosis. Nat Rev Dis Primers. 2018; 4(1):9.
16. Soliman A M, Yang H, Du E X, Kelley C, Winkel C. The direct and indirect costs associated with endometriosis: a systematic literature review. Hum Reprod. 2016; 31(4):712-22.
17. Armour M, Lawson K, Wood A, Smith C A, Abbott J. The cost of illness and economic burden of endometriosis and chronic pelvic pain in Australia: A national online survey. PLoS One. 2019; 14(10):e0223316.
18. Simoens S, Dunselman G, Dirksen C, Hummelshoj L, Bokor A, Brandes I, et al. The burden of endometriosis: costs and quality of life of women with endometriosis and treated in referral centres. Hum Reprod. 2012; 27(5):1292-9.
19. Fuldeore M J, Chwalisz K, Marx S, Wu N, Boulanger L, Ma L, et al. Surgical procedures and their cost estimates among women with newly diagnosed endometriosis: a U S database study. Journal of Medical Economics. 2011; 1(1):115-23.
20. Surrey E, Soliman A M, Trenz H, Blauer-Peterson C, Sluis A. Impact of Endometriosis Diagnostic Delays on Healthcare Resource Utilization and Costs. Adv Ther. 2020; 37(3):1087-99.
21. Nnoaham K E, Hummelshoj L, Kennedy S H, Jenkinson C, Zondervan K T, World Endometriosis Research Foundation Women's Health Symptom Survey C. Developing symptom-based predictive models of endometriosis as a clinical screening tool: results from a multicenter study. Fertil Steril. 2012; 98(3):692-701 e5.
22. Guerriero S, Saba L, Ajossa S, Peddes C, Angiolucci M, Perniciano M, et al. Three-dimensional ultrasonography in the diagnosis of deep endometriosis. Hum Reprod. 2014; 29(6):1189-98.
23. Khan K S, Tryposkiadis K, Tirlapur S A, Middleton L J, Sutton A J, Priest L, et al. MRI versus laparoscopy to diagnose the main causes of chronic pelvic pain in women: a test-accuracy study and economic evaluation. Health technology assessment. 2018; 22(40):1-92.
24. Bermot C, Labauge P, Limot O, Louboutin A, Fauconnier A, Huchon C. Performance of MRI for the detection of anterior pelvic endometriotic lesions. J Gynecol Obstet Hum Reprod. 2018; 47(10):499-503.
25. Vanhie A, O D, Peterse D, Beckers A, Cuellar A, Fassbender A, et al. Plasma miRNAs as biomarkers for endometriosis. Hum Reprod. 2019; 34(9):1650-60.
26. Wang F, Wang H, Jin D, Zhang Y. Serum miR-17, IL-4, and IL-6 levels for diagnosis of endometriosis. Medicine (Baltimore). 2018; 97(24):e10853.
27. Browne A S, Yu J, Huang R P, Francisco A M, Sidell N, Taylor R N. Proteomic identification of neurotrophins in the eutopic endometrium of women with endometriosis. Fertil Steril. 2012; 98(3):713-9.
28. Deitos A, Dussan-Sarria J A, Souza A, Medeiros L, Tarrago Mda G, Sehn F, et al. Clinical Value of Serum Neuroplasticity Mediators in Identifying the Central Sensitivity Syndrome in Patients With Chronic Pain With and Without Structural Pathology. Clin J Pain. 2015; 31(11):959-67.
29. Schega L, Peter B, Brigadski T, Lessmann V, Isermann B, Hamacher D, et al. Effect of intermittent normobaric hypoxia on aerobic capacity and cognitive function in older people. J Sci Med Sport. 2016; 19(11):941-5.
30. Mol B W, Bayram N, Lijmer J G, Wiegerinck M A, Bongers M Y, van der Veen F, et al. The performance of CA-125 measurement in the detection of endometriosis: a meta-analysis. Fertil Steril. 1998; 70(6):1101-8.
31. Chen L, Wang X, Shu J, Xu S, Wu Q, Yu Y. Diagnostic value of serum D-dimer, CA125, and neutrophil-to-lymphocyte ratio in differentiating ovarian cancer and endometriosis. Int J Gynaecol Obstet. 2019; 147(2):212-8.
32. Browne H, Taylor H. HOXA10 expression in ectopic endometrial tissue. Fertil Steril. 2006; 85(5):1386-90.
33. Kim J J, Taylor H S, Lu Z, Ladhani O, Hastings J M, Jackson K S, et al. Altered expression of HOXA10 in endometriosis: potential role in decidualization. Mol Hum Reprod. 2007; 13(5):323-32.
34. Wu Y, Halverson G, Basir Z, Strawn E, Yan P, Guo S W. Aberrant methylation at HOXA10 may be responsible for its aberrant expression in the endometrium of patients with endometriosis. Am J Obstet Gynecol. 2005; 193(2):371-80.
35. Soreide K. Receiver-operating characteristic curve analysis in diagnostic, prognostic and predictive biomarker research. J Clin Pathol. 2009; 62(1):1-5.
36. Wald N J, Bestwick J P. Is the area under an ROC curve a valid measure of the performance of a screening or diagnostic test?J Med Screen. 2014; 21(1):51-6.
37. Haid M, Muschet C, Wahl S, Romisch-Margl W, Prehn C, Moller G, et al. Long-term stability of human plasma metabolites during storage at −80 degrees C. J Proteome Res. 2017.
38. Bennett M J, Santani A B. Carnitine Palmitoyltransferase 1A Deficiency. In: Pagon R A, Adam M P, Bird T D, Dolan C R, Fong C T, Stephens K, editors. GeneReviews. Seattle (WA) 1993.
39. Robinson M D, Smyth G K. Small-sample estimation of negative binomial dispersion, with applications to SAGE data. Biostatistics. 2008; 9(2):321-32.
40. Love M I, Huber W, Anders S. Moderated estimation of fold change and dispersion for RNA-seq data with DESeq2. Genome biology. 2014; 15(12):550.
41. Cheng Y, Xie G, Chen T, Qiu Y, Zou X, Zheng M, et al. Distinct urinary metabolic profile of human colorectal cancer. J Proteome Res. 2012; 11(2):1354-63.
42. Adam J, Brandmaier S, Leonhardt J, Scheerer M F, Mohney R P, Xu T, et al. Metformin Effect on Nontargeted Metabolite Profiles in Patients With Type 2 Diabetes and in Multiple Murine Tissues. Diabetes. 2016; 65(12):3776-85.
43. Irandoost, E., S. Najibi, S. Talebbeigi, and S. Nassiri, Focus on the role of NLRP3 inflammasome in the pathology of endometriosis: a review on molecular mechanisms and possible medical applications. Naunyn Schmiedebergs Arch Pharmacol, 2022.
44. K Vouk, N Hevir, M Ribić-Pucelj, G Haarpaintner, H Scherb, J Osredkar, G Möller, C Prehn, T Lanišnik Rižner, J Adamski, Discovery of phosphatidylcholines and sphingomyelins as biomarkers for ovarian endometriosis. Hum Reprod. 2012(10):2955-65.
45. Griffiths W J, Koal T, Wang Y, Kohl M, Enot D P, Deigner H P (2010) Targeted metabolomics for biomarker discovery. Angew Chem Int Ed Engl 49: 5426-5445, and Koal T, Deigner H P (2010).
46. Koal T, Deigner H P (2010) Challenges in mass spectrometry based targeted metabolomics. Curr Mol Med 10: 216-226

1.-35. (canceled)

36. An ex vivo method of diagnosing endometriosis and/or any subtype thereof in a subject comprising quantifying in a sample obtained from said subject the concentrations of at least three pairs of metabolic biomarkers selected from the pairs consisting of
LysoPC a C17:0, SM(OH) C16:1+Arg, PC ae C36:0+PC ae C38:0, PC ae C40:0,
Arg, PC ae C36:0+lysoPC a C16:0, SM C18:1+PC ae C38:0, PC ae C40:0,
Thr, PC aa C34:3+LysoPC a C17:0, SM(OH) C16:1+Arg, PC ae C36:0,
Thr, PC aa C34:3+Arg, PC ae C36:0+lysoPC a C16:0, SM C18:1,
Arg, PC aa C36:6+LysoPC a C17:0, SM(OH) C16:1+Arg, PC ae C36:0,
LysoPC a C17:0, SM(OH) C16:1+Arg, PC ae C36:0+C18, lysoPC a C14:0,
LysoPC a C16:0, SM C18:1+PC ae C38:0, PC ae C40:0+Ser, PC ae C44:3,
LysoPC a C17:0, SM(OH) C16:1+Arg, PC ae C36:0+Trp, PC ae C38:3,
Arg, PC ae C36:0+lysoPC a C16:0, SM C18:1+C8, PC ae C30:0,
Thr, PC ae C36:5+Arg, PC ae C36:0+lysoPC a C16:0, SM C18:1,
Arg, PC ae C36:0+C18, lysoPC a C14:0+lysoPC a C16:0, SM C18:1,
Arg, PC ae C36:0+C10, PC ae C38:6+lysoPC a C16:0, SM C18:1,
Arg, PC aa C36:6+Arg, PC ae C36:0+lysoPC a C16:0, SM C18:1,
Tyr, PC aa C42:4+C3-DC, C18+PC aa C42:1, SM C22:3,
C3-DC, C18+PC aa C42:1, SM C22:3+C6 (C4:1-DC), SM C16:1,
lysoPC a C16:0, SM(OH) C16:1+PC aa C32:0, SM C18:0+PC aa C32:0, PC aa C38:3,
lysoPC a C16:0, SM(OH) C16:1+PC aa C32:0, SM C18:0+Arg, PC ae C34:0,
C5-M-DC, PC aa C42:5+Arg, PC ae C34:0+lysoPC a C18:2, PC ae C40:6,
lysoPC a C18:2, PC ae C40:4+PC ae C40:6, CPT I ratio+lysoPC a C17:0, SM C18:0,
C4, PC ae C30:2+Arg, PC ae C34:0+lysoPC a C18:2, PC ae C40:6,
PC ae C40:6, CPT I ratio+C4, PC ae C30:2+lysoPC a C18:2, PC ae C40:6,
lysoPC a C18:2, PC ae C40:4+Arg, PC ae C34:0+PC ae C34:1, PC ae C42:0,
Orn, PC ae C38:0+C4, PC aa C38:4+Tyr, PC aa C42:2,
Arg, PC aa C36:6+C5, lysoPC a C17:0+C5, Arg,
Orn, PC ae C38:0+C5, lysoPC a C17:0+C5, Arg,
C0, Gly+Orn, PC ae C38:0+Tyr, PC aa C42:2,
SM C18:0+C5, lysoPC a C17:0+C5, Arg,
C5, lysoPC a C17:0+C5, Arg+Ser, SM(OH) C16:1,
SM C18:0+C0, Gly+Tyr, PC aa C42:2,
Orn, PC ae C38:0+C3, PC ae C40:5+Tyr, PC aa C42:2,
Pro, PC ae C34:0+Orn, PC ae C38:0+Tyr, PC aa C42:2,
C4, Ser+Orn, PC ae C38:0+Tyr, PC aa C42:2,
SM C18:0+Orn, PC ae C38:0+Tyr, PC aa C42:2,
Orn, PC ae C38:0+C4, PC ae C40:3+Tyr, PC aa C42:2,
Orn, PC ae C38:0+PC ae C42:3, SM(OH) C16:1+Tyr, PC aa C42:2,
Orn, PC ae C38:0+Tyr, PC ae C38:0+Tyr, PC aa C42:2,
Gly, SM C24:1+PC aa C32:0, PC aa C40:1+PC aa C36:4, PC aa C38:0,
Gly, PC aa C42:5+PC aa C36:4, PC aa C38:0+C0, SM(OH) C22:2,
PC aa C36:3, PC ae C40:5+lysoPC a C14:0, PC aa C28:1+Met, PC aa C36:3,
PC aa C38:0, PC ae C36:1+Thr, SM (OH) C22:1+lysoPC a C14:0,
PC aa C28:1,
Thr, SM (OH) C22:1+PC aa C28:1, PC ae C34:3+C18:2, PC ae C34:3,
PC aa C36:3, PC ae C40:5+C3, PC ae C34:1+Met, PC aa C36:3,
PC aa C36:3, PC ae C40:5+PC aa C28:1, PC ae C34:3+Gly, PC ae C36:1,
PC aa C38:0, PC ae C36:1+C18:2, PC ae C34:3+Met, PC aa C36:3,
PC aa C38:0, PC ae C36:1+C10:1, PC aa C36:1+PC ae C38:3, SM C18:1,
PC aa C38:0, PC ae C36:1+Gly, PC ae C36:1+C3, PC ae C34:1,
PC aa C38:0, PC ae C36:1+C12-DC, C14:2+PC ae C38:3, SM C18:1,
PC aa C36:3, PC ae C40:5+PC aa C38:3, PC ae C44:5+Met, PC aa C36:3,
PC aa C38:0, PC ae C36:1+PC ae C38:3, SM C18:1+Met, PC aa C36:3,
PC aa C28:1, PC ae C34:3+C18:2, PC ae C34:3+C4, C5:1,
PC aa C36:3, PC ae C40:5+lysoPC a C20:4, PC ae C32:1+Met, PC aa C36:3,
PC aa C36:3, PC ae C40:5+lysoPC a C20:4, PC aa C32:3+Met, PC aa C36:3,
C10, PC aa C36:6+Pro, PC ae C34:0+PC ae C42:3, SM(OH) C16:1,
C10, PC aa C36:6+PC ae C42:3, SM(OH) C16:1+C6:1, lysoPC a C20:4,
C10, PC aa C36:6+PC ae C42:3, SM(OH) C16:1+lysoPC a C20:4, PC ae C40:2,
Ser, PC aa C38:3+C10, PC aa C36:6+PC ae C42:3, SM(OH) C16:1,
C10, lysoPC a C18:1+C10, PC aa C36:6+PC ae C42:3, SM(OH) C16:1,
C10, PC aa C36:6+lysoPC a C24:0, PC ae C42:3+PC ae C42:3, SM(OH) C16:1,
C10, PC aa C36:6+lysoPC a C18:1, PC aa C36:1+PC ae C42:3, SM(OH) C16:1,
C10, PC aa C36:6+Gly, PC ae C34:1+PC ae C42:3, SM(OH) C16:1,
Gln, PC ae C30:2+C10, PC aa C36:6+PC ae C42:3, SM(OH) C16:1,
Pro, PC ae C34:0+lysoPC a C24:0, PC ae C42:3+PC ae C42:3, SM(OH) C16:1,
C10:1, lysoPC a C24:0+lysoPC a C24:0, PC ae C42:3+PC ae C42:3, SM(OH) C16:1,
PC ae C44:3, CPT.I.ratio+PC ae C34:0, PC ae C40:3+C16:2-OH, SM C20:2, and
PC ae C44:6, SM C22:3+PC ae C34:0, PC ae C40:3+C10:1, C14:2-OH.

37. The method according to claim 36, which comprises a) quantifying in a sample obtained from said subject the concentrations of at least three pairs of metabolic biomarkers, and b) obtaining a diagnostic score using a generalized linear model (GLM).

38. An ex vivo method of diagnosing endometriosis and/or any subtype thereof in a subject comprising quantifying in a sample obtained from said subject the concentrations of at least three pairs of metabolic biomarkers, and obtaining a diagnostic score using a generalized linear model (GLM) selected from the group consisting of:
LysoPC a C17:0_div_by_SM(OH) C16:1+Arg_div_by_PC ae C36:0+PC ae C38:0_div_by_PC ae C40:0,
Arg_div_by_PC ae C36:0+lysoPC a C16:0_div_by_SM C18:1+PC ae C38:0_div_by_PC ae C40:0,
Thr_div_by_PC aa C34:3+LysoPC a C17:0_div_by_SM(OH) C16:1+Arg_div_by_PC ae C36:0,
Thr_div_by_PC aa C34:3+Arg_div_by_PC ae C36:0+lysoPC a C16:0_div_by_SM C18:1,
Arg_div_by_PC aa C36:6+LysoPC a C17:0_div_by_SM(OH) C16:1+Arg_div_by_PC ae C36:0,
LysoPC a C17:0_div_by_SM(OH) C16:1+Arg_div_by_PC ae C36:0+C18_div_by_lysoPC a C14:0,
LysoPC a C16:0_div_by_SM C18:1+PC ae C38:0_div_by_PC ae C40:0+Ser_div_by_PC ae C44:3,
LysoPC a C17:0_div_by_SM(OH) C16:1+Arg_div_by_PC ae C36:0+Trp_div_by_PC ae C38:3,
Arg_div_by_PC ae C36:0+lysoPC a C16:0_div_by_SM C18:1+C8_div_by_PC ae C30:0,
Thr_div_by_PC ae C36:5+Arg_div_by_PC ae C36:0+lysoPC a C16:0_div_by_SM C18:1,
Arg_div_by_PC ae C36:0+C18_div_by_lysoPC a C14:0+lysoPC a C16:0_div_by_SM C18:1,
Arg_div_by_PC ae C36:0+C10_div_by_PC ae C38:6+lysoPC a C16:0_div_by_SM C18:1,
Arg_div_by_PC aa C36:6+Arg_div_by_PC ae C36:0+lysoPC a C16:0_div_by_SM C18:1,
Tyr_div_by_PC aa C42:4+C3-DC_div_by_C18+PC aa C42:1_div_by_SM C22:3,
C3-DC_div_by_C18+PC aa C42:1_div_by_SM C22:3+C6 (C4:1-DC)_div_by_SM C16:1,
lysoPC a C16:0_div_by_SM(OH) C16:1+PC aa C32:0_div_by_SM C18:0+PC aa C32:0_div_by_PC aa C38:3,
lysoPC a C16:0_div_by_SM(OH) C16:1+PC aa C32:0_div_by_SM C18:0+Arg_div_by_PC ae C34:0,
C5-M-DC_div_by_PC aa C42:5+Arg_div_by_PC ae C34:0+lysoPC a C18:2_div_by_PC ae C40:6,
lysoPC a C18:2_div_by_PC ae C40:4+PC ae C40:6_div_by_CPT I ratio+lysoPC a C17:0_div_by_SM C18:0,
C4_div_by_PC ae C30:2+Arg_div_by_PC ae C34:0+lysoPC a C18:2_div_by_PC ae C40:6,
PC ae C40:6_div_by_CPT I ratio+C4_div_by_PC ae C30:2+lysoPC a C18:2_div_by_PC ae C40:6,
lysoPC a C18:2_div_by_PC ae C40:4+Arg_div_by_PC ae C34:0+PC ae C34:1_div_by_PC ae C42:0,
Orn_div_by_PC ae C38:0+C4_div_by_PC aa C38:4+Tyr_div_by_PC aa C42:2,
Arg_div_by_PC aa C36:6+C5_div_by_lysoPC a C17:0+C5_div_by_Arg,
Orn_div_by_PC ae C38:0+C5_div_by_lysoPC a C17:0+C5_div_by_Arg,
C0_div_by_Gly+Orn_div_by_PC ae C38:0+Tyr_div_by_PC aa C42:2,
SM C18:0+C5_div_by_lysoPC a C17:0+C5_div_by_Arg,
C5_div_by_lysoPC a C17:0+C5_div_by_Arg+Ser_div_by_SM(OH) C16:1,
SM C18:0+C0_div_by_Gly+Tyr_div_by_PC aa C42:2,
Orn_div_by_PC ae C38:0+C3_div_by_PC ae C40:5+Tyr_div_by_PC aa C42:2,
Pro_div_by_PC ae C34:0+Orn_div_by_PC ae C38:0+Tyr_div_by_PC aa C42:2,
C4_div_by_Ser+Orn_div_by_PC ae C38:0+Tyr_div_by_PC aa C42:2,
SM C18:0+Orn_div_by_PC ae C38:0+Tyr_div_by_PC aa C42:2,
Orn_div_by_PC ae C38:0+C4_div_by_PC ae C40:3+Tyr_div_by_PC aa C42:2,
Orn_div_by_PC ae C38:0+PC ae C42:3_div_by_SM(OH) C16:1+Tyr_div_by_PC aa C42:2,
Orn_div_by_PC ae C38:0+Tyr_div_by_PC ae C38:0+Tyr_div_by_PC aa C42:2,
Gly_div_by_SM C24:1+PC aa C32:0_div_by_PC aa C40:1+PC aa C36:4_div_by_PC aa C38:0,
Gly_div_by_PC aa C42:5+PC aa C36:4_div_by_PC aa C38:0+C0_div_by_SM(OH) C22:2,
PC aa C36:3_div_by_PC ae C40:5+lysoPC a C14:0_div_by_PC aa C28:1+Met_div_by_PC aa C36:3, PC aa C38:0_div_by_PC ae C36:1+Thr_div_by_SM (OH) C22:1+lysoPC a C14:0_div_by_PC aa C28:1,
Thr_div_by_SM (OH) C22:1+PC aa C28:1_div_by_PC ae C34:3+C18:2_div_by_PC ae C34:3,
PC aa C36:3_div_by_PC ae C40:5+C3_div_by_PC ae C34:1+Met_div_by_PC aa C36:3,
PC aa C36:3_div_by_PC ae C40:5+PC aa C28:1_div_by_PC ae C34:3+Gly_div_by_PC ae C36:1,
PC aa C38:0_div_by_PC ae C36:1+C18:2_div_by_PC ae C34:3+Met_div_by_PC aa C36:3,
PC aa C38:0_div_by_PC ae C36:1+C10:1_div_by_PC aa C36:1+PC ae C38:3_div_by_SM C18:1,
PC aa C38:0_div_by_PC ae C36:1+Gly_div_by_PC ae C36:1+C3_div_by_PC ae C34:1,
PC aa C38:0_div_by_PC ae C36:1+C12-DC_div_by_C14:2+PC ae C38:3_div_by_SM C18:1,
PC aa C36:3_div_by_PC ae C40:5+PC aa C38:3_div_by_PC ae C44:5+Met_div_by_PC aa C36:3,
PC aa C38:0_div_by_PC ae C36:1+PC ae C38:3_div_by_SM C18:1+Met_div_by_PC aa C36:3,
PC aa C28:1_div_by_PC ae C34:3+C18:2_div_by_PC ae C34:3+C4_div_by_C5:1,
PC aa C36:3_div_by_PC ae C40:5+lysoPC a C20:4_div_by_PC ae C32:1+Met_div_by_PC aa C36:3,
PC aa C36:3_div_by_PC ae C40:5+lysoPC a C20:4_div_by_PC aa C32:3+Met_div_by_PC aa C36:3,
C10_div_by_PC aa C36:6+Pro_div_by_PC ae C34:0+PC ae C42:3_div_by_SM(OH) C16:1,
C10_div_by_PC aa C36:6+PC ae C42:3_div_by_SM(OH) C16:1+C6:1_div_by_lysoPC a C20:4,
C10_div_by_PC aa C36:6+PC ae C42:3_div_by_SM(OH) C16:1+lysoPC a C20:4_div_by_PC ae C40:2,
Ser_div_by_PC aa C38:3+C10_div_by_PC aa C36:6+PC ae C42:3_div_by_SM(OH) C16:1,
C10_div_by_lysoPC a C18:1+C10_div_by_PC aa C36:6+PC ae C42:3_div_by_SM(OH) C16:1,
C10_div_by_PC aa C36:6+lysoPC a C24:0_div_by_PC ae C42:3+PC ae C42:3_div_by_SM(OH) C16:1,
C10_div_by_PC aa C36:6+lysoPC a C18:1_div_by_PC aa C36:1+PC ae C42:3_div_by_SM(OH) C16:1,
C10_div_by_PC aa C36:6+Gly_div_by_PC ae C34:1+PC ae C42:3_div_by_SM(OH) C16:1,
Gln_div_by_PC ae C30:2+C10_div_by_PC aa C36:6+PC ae C42:3_div_by_SM(OH) C16:1,
Pro_div_by_PC ae C34:0+lysoPC a C24:0_div_by_PC ae C42:3+PC ae C42:3_div_by_SM(OH) C16:1,
C10:1_div_by_lysoPC a C24:0+lysoPC a C24:0_div_by_PC ae C42:3+PC ae C42:3_div_by_SM(OH) C16:1,
PC ae C44:3_div_by_CPT.I.ratio+PC ae C34:0_div_by_PC ae C40:3+C16:2-OH_div_by_SM C20:2, and
PC ae C44:6_div_by_SM C22:3+PC ae C34:0_div_by_PC ae C40:3+C10:1_div_by_C14:2-OH.

39. The method according to claim 38, wherein the generalized linear modelling comprises i) determining the ratio of the concentrations for each of the at least three pairs and ii) calculating the sum of the obtained ratios (value for case).

40. The method according to claim 39, wherein the generalized linear modelling (GLM) further comprises iii) obtaining a diagnostic score (DxS) calculated by forming the quotient between a predetermined reference value obtained from healthy subjects (value for control) and the sum of the obtained ratios (value for case)
$$DxS = log 2 ( \frac{predetermined reference value ( value for control )}{sum of the obtained ratios ( value for case )} )$$
wherein said subject is diagnosed of having endometriosis or a sub-type thereof if the diagnostic score is different from zero (“0”), such as outside of the range 0±0.03.

41. The method according to claim 36, comprising determining whether the subject is suffering from any type of endometriosis.

42. The method according to claim 41, comprising
A1) quantifying in a sample obtained from said subject the concentrations of at least three pairs of metabolic biomarkers selected from the group of pairs consisting of:
LysoPC a C17:0, SM(OH) C16:1+Arg, PC ae C36:0+PC ae C38:0,
PC ae C40:0,
Arg, PC ae C36:0+lysoPC a C16:0, SM C18:1+PC ae C38:0, PC ae C40:0,
Thr, PC aa C34:3+LysoPC a C17:0, SM(OH) C16:1+Arg, PC ae C36:0,
Thr, PC aa C34:3+Arg, PC ae C36:0+lysoPC a C16:0, SM C18:1,
Arg, PC aa C36:6+LysoPC a C17:0, SM(OH) C16:1+Arg, PC ae C36:0
LysoPC a C17:0, SM(OH) C16:1+Arg, PC ae C36:0+C18, lysoPC a C14:0,
LysoPC a C16:0, SM C18:1+PC ae C38:0, PC ae C40:0+Ser, PC ae C44:3,
LysoPC a C17:0, SM(OH) C16:1+Arg, PC ae C36:0+Trp, PC ae C38:3,
Arg, PC ae C36:0+lysoPC a C16:0, SM C18:1+C8, PC ae C30:0,
Thr, PC ae C36:5+Arg, PC ae C36:0+lysoPC a C16:0, SM C18:1,
Arg, PC ae C36:0+C18, lysoPC a C14:0+lysoPC a C16:0, SM C18:1,
Arg, PC ae C36:0+C10, PC ae C38:6+lysoPC a C16:0, SM C18:1,
Arg, PC aa C36:6+Arg, PC ae C36:0+lysoPC a C16:0, SM C18:1,
Tyr, PC aa C42:4+C3-DC, C18+PC aa C42:1, SM C22:3, and
C3-DC, C18+PC aa C42:1, SM C22:3+C6 (C4:1-DC), SM C16:1; and
B1) obtaining a diagnostic score using a generalized linear model (GLM).

43. The method according to claim 38, comprising determining whether the subject is suffering from peritoneal endometriosis.

44. The method according to claim 43, comprising
A2) quantifying in a sample obtained from said subject the concentrations of at least three pairs of metabolic biomarkers selected from the group of pairs consisting of
lysoPC a C16:0, SM(OH) C16:1+PC aa C32:0, SM C18:0+PC aa C32:0, PC aa C38:3,
lysoPC a C16:0, SM(OH) C16:1+PC aa C32:0, SM C18:0+Arg, PC ae C34:0,
C5-M-DC, PC aa C42:5+Arg, PC ae C34:0+lysoPC a C18:2, PC ae C40:6,
lysoPC a C18:2, PC ae C40:4+PC ae C40:6, CPT I ratio+lysoPC a C17:0, SM C18:0,
C4, PC ae C30:2+Arg, PC ae C34:0+lysoPC a C18:2, PC ae C40:6,
PC ae C40:6, CPT I ratio+C4, PC ae C30:2+lysoPC a C18:2, PC ae C40:6, and
lysoPC a C18:2, PC ae C40:4+Arg, PC ae C34:0+PC ae C34:1, PC ae C42:0; and
B2) obtaining a diagnostic score using a generalized linear model (GLM).

45. The method according to claim 36, comprising determining whether the subject is suffering from peritoneal mixed endometriosis.

46. The method according to claim 45, comprising
A3) quantifying in a sample obtained from said subject the concentrations of at least three pairs of metabolic biomarkers selected from the group of pairs consisting of
Orn, PC ae C38:0+C4, PC aa C38:4+Tyr, PC aa C42:2,
Arg, PC aa C36:6+C5, lysoPC a C17:0+C5, Arg,
Orn, PC ae C38:0+C5, lysoPC a C17:0+C5, Arg,
C0, Gly+Orn, PC ae C38:0+Tyr, PC aa C42:2,
SM C18:0+C5, lysoPC a C17:0+C5, Arg,
C5, lysoPC a C17:0+C5, Arg+Ser, SM(OH) C16:1,
SM C18:0+C0, Gly+Tyr, PC aa C42:2,
Orn, PC ae C38:0+C3, PC ae C40:5+Tyr, PC aa C42:2,
Pro, PC ae C34:0+Orn, PC ae C38:0+Tyr, PC aa C42:2,
C4, Ser+Orn, PC ae C38:0+Tyr, PC aa C42:2,
SM C18:0+Orn, PC ae C38:0+Tyr, PC aa C42:2,
Orn, PC ae C38:0+C4, PC ae C40:3+Tyr, PC aa C42:2,
Orn, PC ae C38:0+PC ae C42:3, SM(OH) C16:1+Tyr, PC aa C42:2,
Orn, PC ae C38:0+Tyr, PC ae C38:0+Tyr, PC aa C42:2,
Gly, SM C24:1+PC aa C32:0, PC aa C40:1+PC aa C36:4, PC aa C38:0, and
Gly, PC aa C42:5+PC aa C36:4, PC aa C38:0+C0, SM(OH) C22:2
B3) performing generalized linear modelling (GLM).

47. The method according to claim 36, comprising determining whether the subject is suffering from ovarian endometriosis.

48. The method according to claim 47, comprising
A4) quantifying in a sample obtained from said subject the concentrations of at least three pairs of metabolic biomarkers selected from the group of pairs consisting of
PC aa C36:3, PC ae C40:5+lysoPC a C14:0, PC aa C28:1+Met, PC aa C36:3,
PC aa C38:0, PC ae C36:1+Thr, SM (OH) C22:1+lysoPC a C14:0, PC aa C28:1,
Thr, SM (OH) C22:1+PC aa C28:1, PC ae C34:3+C18:2, PC ae C34:3,
PC aa C36:3, PC ae C40:5+C3, PC ae C34:1+Met, PC aa C36:3,
PC aa C36:3, PC ae C40:5+PC aa C28:1, PC ae C34:3+Gly, PC ae C36:1,
PC aa C38:0, PC ae C36:1+C18:2, PC ae C34:3+Met, PC aa C36:3,
PC aa C38:0, PC ae C36:1+C10:1, PC aa C36:1+PC ae C38:3, SM C18:1,
PC aa C38:0, PC ae C36:1+Gly, PC ae C36:1+C3, PC ae C34:1,
PC aa C38:0, PC ae C36:1+C12-DC, C14:2+PC ae C38:3, SM C18:1,
PC aa C36:3, PC ae C40:5+PC aa C38:3, PC ae C44:5+Met, PC aa C36:3,
PC aa C38:0, PC ae C36:1+PC ae C38:3, SM C18:1+Met, PC aa C36:3,
PC aa C28:1, PC ae C34:3+C18:2, PC ae C34:3+C4, C5:1,
PC aa C36:3, PC ae C40:5+lysoPC a C20:4, PC ae C32:1+Met, PC aa C36:3, and
PC aa C36:3, PC ae C40:5+lysoPC a C20:4, PC aa C32:3+Met, PC aa C36:3; and
B4) performing generalized linear modelling (GLM).

49. The method according to claim 36, comprising determining whether the subject is suffering from ovarian mixed endometriosis.

50. The method according to claim 49, comprising
A5) quantifying in a sample obtained from said subject the concentrations of at least three pairs of metabolic biomarkers selected from the group of pairs consisting of
C10, PC aa C36:6+Pro, PC ae C34:0+PC ae C42:3, SM(OH) C16:1,
C10, PC aa C36:6+PC ae C42:3, SM(OH) C16:1+C6:1, lysoPC a C20:4,
C10, PC aa C36:6+PC ae C42:3, SM(OH) C16:1+lysoPC a C20:4, PC ae C40:2,
Ser, PC aa C38:3+C10, PC aa C36:6+PC ae C42:3, SM(OH) C16:1,
C10, lysoPC a C18:1+C10, PC aa C36:6+PC ae C42:3, SM(OH) C16:1,
C10, PC aa C36:6+lysoPC a C24:0, PC ae C42:3+PC ae C42:3, SM(OH) C16:1,
C10, PC aa C36:6+lysoPC a C18:1, PC aa C36:1+PC ae C42:3, SM(OH) C16:1,
C10, PC aa C36:6+Gly, PC ae C34:1+PC ae C42:3, SM(OH) C16:1,
Gln, PC ae C30:2+C10, PC aa C36:6+PC ae C42:3, SM(OH) C16:1,
Pro, PC ae C34:0+lysoPC a C24:0, PC ae C42:3+PC ae C42:3, SM(OH) C16:1,
C10:1, lysoPC a C24:0+lysoPC a C24:0, PC ae C42:3+PC ae C42:3, SM(OH) C16:1,
PC ae C44:3, CPT.I.ratio+PC ae C34:0, PC ae C40:3+C16:2-OH, SM C20:2, and
PC ae C44:6, SM C22:3+PC ae C34:0, PC ae C40:3+C10:1, C14:2-OH; and
B5) performing generalized linear modelling (GLM).

51. The method according to claim 36, wherein the sample is selected from blood, serum, plasma, saliva, urine, cerebrospinal fluid, condensates from respiratory air, tears, mucosal tissue, mucus, vaginal tissue, endometrium, eutopic endometrium, skin, hair or hair follicle.

52. The method according to claim 36, wherein the sample is blood, serum or plasma.

53. The method according to claim 36, wherein the subject is a human subject.

54. The method according to claim 53, wherein the human subject is a female.
